(19) United States
(12) Patent Application Publication
(10) Pub. No.: US 2022/0226350 A1
(43) Pub. Date: Jul. 21, 2022

(54) PHARMACEUTICAL COMPOSITIONS COMPRISING A FXR AGONIST AND A FIBRATE FOR USE IN THE TREATMENT OF CHOLESTATIC LIVER DISEASE

(71) Applicant: Intercept Pharmaceuticals, Inc., New York, NY (US)

(72) Inventors: Leigh A. MACCONELL, Encinitas, CA (US); Richard PENCEK, San Diego, CA (US)

(73) Assignee: Intercept Pharmaceuticals, Inc., New York, NY (US)

(21) Appl. No.: 17/615,186

(22) PCT Filed: May 29, 2020
(86) PCT No.: PCT/US2020/035353

§ 371 Date: Nov. 30, 2021

Related U.S. Application Data
(60) Provisional application No. 62/854,859, filed on May 30, 2019.

(51) Int. Cl. A61K 31/575; A61K 31/195; A61P 1/16

(52) U.S. Cl. CPC A61K 31/575; A61P 1/16; A61K 31/195

(57) ABSTRACT

The present invention relates to a pharmaceutical composition comprising a combination of an FXR agonist and a fibrate. Also disclosed is use of the combination for the treatment, amelioration or prevention of an FXR mediated disease or condition, such as primary biliary cholangitis (PBC).

### RELATED APPLICATIONS

This application claims priority to, and the benefit of, U.S. Provisional Application No. 62/854,859, filed on May 30, 2019, the contents of which are hereby incorporated by reference in their entirety.

### BACKGROUND

Primary biliary cholangitis (PBC) is a serious, life-threatening, cholestatic liver disease of unknown etiology that, without treatment, frequently progresses to hepatic fibrosis and eventual cirrhosis, hepatic decompensation, and necessitates liver transplantation or results in death. Subjects with advanced PBC disease are also predisposed to hepatocellular carcinoma. PBC is a rare disease with reported prevalence in the United States (US) of about 40.2/100 000. PBC disproportionately affects women more than men by approximately 10:1 and is typically diagnosed in patients between 40 and 60 years of age.

Historically, the only approved drug therapy for PBC has been the bile acid ursodeoxycholic acid (UDCA), a physiological constituent of human bile. While UDCA therapy had a marked effect on the treatment of PBC, up to 50% of patients showed a suboptimal response or no response to UDCA. Such patients were at significantly increased risk of a poor clinical outcome due to PBC disease progression.

Fibrates have anticholestatic, anti-inflammatory, and antifibrotic effects and have recently shown the potential to further improve the biochemical markers of PBC. The mechanisms that underlie these effects are complementary, and largely mediated through activation of peroxisome proliferator activated receptors. Fibrate treatment has been found promising in ameliorating liver biochemical tests in UDCA unresponsive patients, either as monotherapy or in combination with UDCA. Bezafibrate (BZF) has been identified as a potential anticholestatic agent for the treatment of PBC with an inadequate response to UDCA.

Obeticholic acid (OCA), a farnesoid X receptor (FXR) agonist and modified bile acid derived from the primary human bile acid chenodeoxycholic acid (CDCA), was developed for the treatment of PBC and to provide patients who have an inadequate response to or poor tolerance of UDCA, a novel treatment option that was safe and effective. OCA is approved under the tradename OCALIVA by the US Food and Drug Administration (FDA), European Medicines Agency (EMA; conditional approval), Health Canada, and other regulatory agencies for the treatment of PBC in combination with UDCA in adults with inadequate response to UDCA, or as monotherapy in adults unable to tolerate UDCA. However, OCA monotherapy can cause itching (pruritus) as an adverse event.

There is a need for an improved therapy for the treatment of cholestatic diseases and conditions, e.g., PBC, especially in patients who have an inadequate response to or cannot tolerate existing therapies.

### SUMMARY

The present invention relates to a pharmaceutical composition comprising a combination of an FXR agonist, a fibrate, and optionally one or more pharmaceutically acceptable carriers.

The present invention also relates to the therapeutic use of the pharmaceutical compositions of the present invention.

The present invention relates to the therapeutic use of the pharmaceutical compositions comprising a combination of an FXR agonist, a fibrate, and optionally one or more pharmaceutically acceptable carriers.

In one embodiment, the FXR agonist is a compound of formula A:

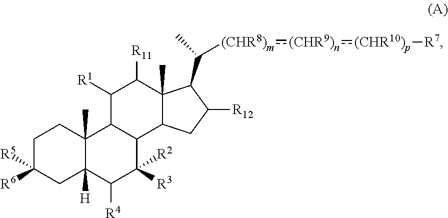

or a pharmaceutically acceptable salt, solvate, amino acid, sulfate or glucuronide conjugate, or prodrug thereof, wherein R₁, R₂, R₃, R₄, R₅, R₆, R₇, R₈, R₉, R₁₀, R₁₁, and R₁₂ are as defined herein.

The present invention also relates to methods for treating or preventing an FXR mediated disease or condition, reducing the level of a liver enzyme, or inhibiting or reversing fibrosis comprising administering a therapeutically effective amount of a pharmaceutical composition comprising a combination of an FXR agonist, a fibrate, and optionally one or more pharmaceutically acceptable carriers to a subject in need thereof.

The present invention also relates to use of a pharmaceutical composition comprising a combination of an FXR agonist, a fibrate, and optionally one or more pharmaceutically acceptable carriers for treating or preventing an FXR mediated disease or condition, reducing the level of a liver enzyme, or inhibiting or reversing fibrosis.

The present invention also relates to use of a pharmaceutical composition of the present invention in the manufacture of a medicament for treating or preventing an FXR mediated disease or condition, reducing the level of a liver enzyme, or inhibiting or reversing fibrosis.

The present invention relates to the treatment of liver diseases or conditions comprising administering a pharmaceutical composition comprising a combination of an FXR agonist, a fibrate, and optionally one or more pharmaceutically acceptable carriers to a subject in need thereof.

The compositions and methods of the present invention address unmet needs in the treatment or prevention of an FXR mediated disease or disorder (e.g., PBC).

### BRIEF DESCRIPTION OF THE DRAWINGS

FIG. 1 is a diagram showing the study design of a double-blind treatment period, where BZF=bezafibrate; DB=double-blind; EODB=end of DB; OCA=obeticholic acid; QD=once daily; UDCA=ursodeoxycholic acid. Subjects taking UDCA at the time of enrollment remain on their stable dose of UDCA during the study. The DB treatment continues until all subjects have completed Week 12 in the DB Treatment Period.

FIG. 2 is a diagram showing the study design diagram of a long-term safety extension period, where BZF=bezafibrate; EOS=end of study/end of LTSE Period; OCA=obeticholic acid; LTSE=long-term safety extension; QD=once daily; UDCA=ursodeoxycholic acid. Subjects taking UDCA at the time of reconsent remain on their stable dose of UDCA during the study.

FIG. 3 is a diagram showing the study design for the double-blind and LTSE treatment periods, where BZF=bezafibrate; DB=double-blind; EODB=end of DB; EOS=end of study/end of LTSE Period; LTSE=long-term safety extension; OCA=obeticholic acid; QD=once daily; UDCA=ursodeoxycholic acid; and placebo=either OCA or BZF tablets.

### DETAILED DESCRIPTION

The present application is directed to a pharmaceutical composition comprising an FXR agonist, a fibrate, and optionally one or more pharmaceutically acceptable carriers and the methods of use thereof. The present disclosure relates to a concomitant use of an FXR agonist, such as OCA, and a fibrate, such as BZF, for preventing, ameliorating or treating an FXR mediated disease or disorder (e.g., PBC). The present disclosure also relates to a concomitant use of an FXR agonist, such as OCA, and a fibrate, such as BZF to improve efficacy and tolerability compared to the existing treatments (e.g., the UDCA mono or combination therapies or treatment with OCA alone).

In one aspect, an FXR agonist is a compound of formula A:

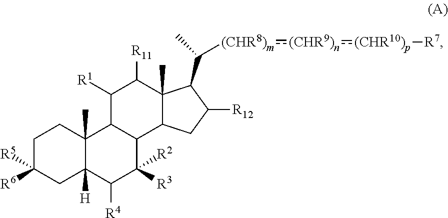

or a pharmaceutically acceptable salt, solvate, amino acid, sulfate or glucuronide conjugate, or prodrug thereof, wherein:
R¹ is OH, alkoxy, or oxo;
R² and R³ are each independently H, OH, OSO₃H, OCOCH₃, OPO₃H₂, halogen, or alkyl optionally substituted with one or more halogen or OH, or R² and R³ taken together with the carbon atom to which they are attached form a carbonyl;

R⁴ is H, halogen, alkyl optionally substituted with one or more halogen or OH, alkenyl, or alkynyl;

R⁵ and R⁶ are each independently H, OH, OSO₃H, OCOCH₃, OPO₃H₂, halogen, or alkyl optionally substituted with one or more halogen or OH, or R⁵ and R⁶ taken together with the carbon atom to which they are attached form a carbonyl;

R⁷ is OH, OSO₃H, SO₃H, OSO₂NH₂, SO₂NH₂, OPO₃H₂, PO₃H₂, CO₂H, C(O)NHOH, NH(CH₂)₂SO₃H, NHCH₂CO₂H, tetrazolyl, oxadiazolyl, thiadiazolyl, 5-oxo-1,2,4-oxadiazolyl, 5-oxo-1,2,4-thiadiazolyl, oxazolidine-dionyl, thiazolidine-dionyl, 3-hydroxyisoxazolyl, 3-hydroxyisothiazolyl, pyrimidine, 3,5-difluoro-4-hydroxyphenyl or 2,4-difluoro-3-hydroxyphenyl;

R⁸, R⁹, and R¹⁰ are each independently H, OH, halogen, or alkyl optionally substituted with one or more halogen or OH, or R⁸ and R⁹ taken together with the carbon atoms to which they are attached form a 3- to 6-membered carbocyclic or heterocyclic ring comprising 1 or 2 heteroatoms selected from N, O, and S, or R⁹ and R¹⁰ taken together with the carbon atoms to which they are attached form a 3- to 6-membered carbocyclic or heterocyclic ring comprising 1 or 2 heteroatoms selected from N, O, and S;
R₁₁ and R₁₂ are each independently H or OH;
m is 0, 1, or 2;
n is 0 or 1; and
p is 0 or 1.

In further aspects, the composition includes a compound of formula A, wherein R₁, R₁₁, and R₁₂ are hydrogen and R₄ is alkyl optionally substituted with one or more halogen or OH, alkenyl, or alkynyl. In further aspects, the composition includes a compound of formula A, wherein R₁ is hydroxy (e.g., alpha- or beta-hydroxy), R₁₁, and R₁₂ are hydrogen and R₄ is alkyl optionally substituted with one or more halogen or OH, alkenyl, or alkynyl. In a further example, the composition includes a compound of formula A, wherein R₄ is unsubstituted C₁-C₆ alkyl. In one aspect, the composition includes a compound of formula A, wherein R₄ is unsubstituted C₁-C₃ alkyl. In one aspect, the composition includes a compound of formula A, wherein R₄ is selected from methyl, ethyl, and propyl. In one aspect, the composition includes a compound of formula A, wherein R₄ is ethyl.

In a further aspect, the composition includes a compound of formula A, wherein R₇ is selected from C(O)OH, C(O)NH(CH₂)ₘSO₃H, and C(O)NH(CH₂)ₙCO₂H. In one aspect, the composition includes a compound of formula A, wherein R₇ is selected from C(O)OH, C(O)NH(CH₂)SO₃H, C(O)NH(CH₂)CO₂H, C(O)NH(CH₂)₂SO₃H, C(O)NH(CH₂)₂CO₂H. In one aspect, the composition includes a compound of formula A, wherein R₇ is C(O)OH. In one aspect, the composition includes a compound of formula A, wherein R₇ is OSO₃H. In one aspect, the composition includes a compound of formula A, wherein the compound is a pharmaceutically acceptable salt. The pharmaceutically acceptable salt can be any salt. In one aspect, the composition includes a compound of formula A, wherein R₇ is OSO₃⁻Na⁺. In one aspect, the composition includes a compound of formula A, wherein R₇ is OSO₃⁻NHEt₃⁺. In one aspect, the amino acid conjugate is a glycine conjugate. In one aspect, the amino acid conjugate is a taurine conjugate.

In yet another aspect, the composition includes a compound of formula A, wherein R₇ is selected from OH, NH(CH₂)SO₃H, NH(CH₂)CO₂H, NH(CH₂)₂SO₃H, and NH(CH₂)₂CO₂H.

In one aspect, the compound of formula A is a compound of formula 1 (also referred to herein as Compound 1, or obeticholic acid):

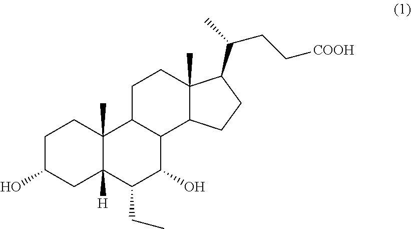 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]

or a pharmaceutically acceptable salt or amino acid conjugate thereof.

In further aspect, the compound of formula 1 is

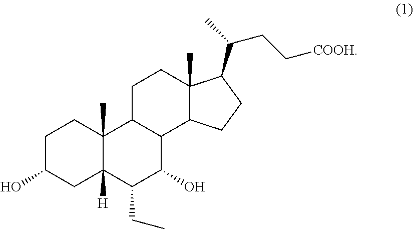 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]

In a further aspect, the compound of formula A is a compound of formula 2 (also referred to herein as Compound 2):

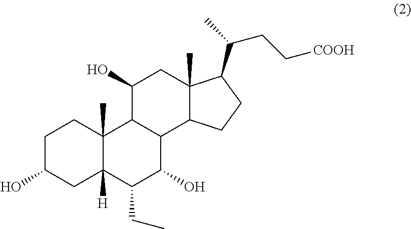 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]

or a pharmaceutically acceptable salt or amino acid conjugate thereof.

In further aspect, the compound of formula 2 is

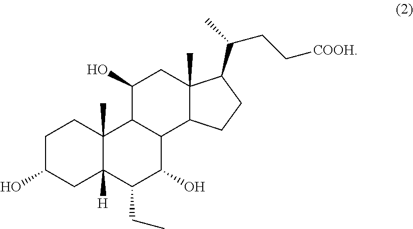 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]

In a further aspect, the compound of formula A is a compound of formula 3 (also referred to herein as Compound 3):

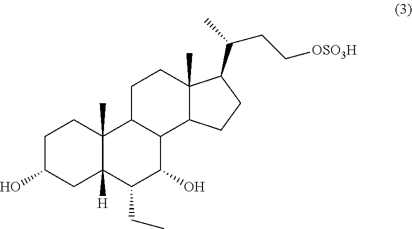

or a pharmaceutically acceptable salt thereof.

In further aspect, the compound of formula 3 is

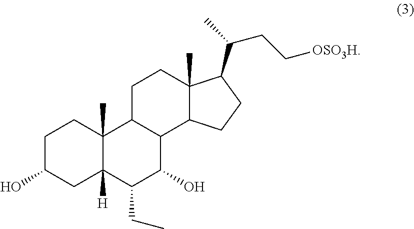

In yet a further example, the composition includes a compound of formula 3 which is a pharmaceutically acceptable salt selected from compound 3a and 3b (also referred to herein as Compound 3a and Compound 3b):

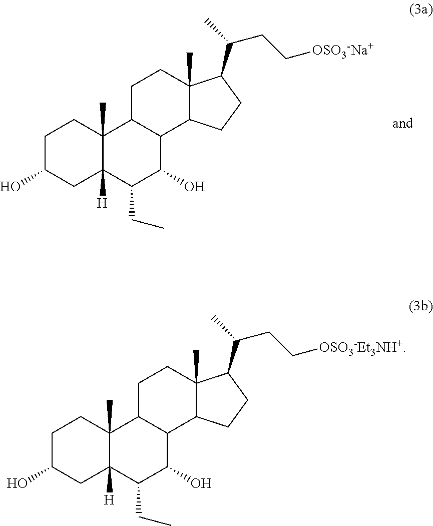

Compounds of formulae 1, 2, 3, 3a and 3b are subsets of compounds of formula A.

The present application also describes the pharmaceutical compositions, packs or kits, and therapeutic uses of the combination.

One of the problems to be solved by the present invention is the identification of combination therapies for the treatment or prevention of conditions related to elevated concentrations of circulating lipid compounds (such as cholesterol and triglycerides) in the blood, e.g., a cholestatic liver condition such as PBC, as well as for the reduction of circulating lipid compounds (e.g., cholesterol, LDL, and triglycerides) in the blood, and for the reduction of bilirubin and/or liver enzymes, such as alkaline phosphatase (ALP, AP, or Alk Phos), alanine aminotransferase (ALT), aspartate aminotransferase (AST), gamma-glutamyl transpeptidase (GGT), lactate dehydrogenase (LDH), and 5′ nucleotidase. Although drugs for conditions related to elevated lipid levels and/or liver enzyme levels are available, these drugs are often not suitable for many patients for a variety of reasons. For example, certain drugs are ineffective for patients who have developed drug resistance, such as in the case of patients resistant to ursodeoxycholic acid. Some drugs may be inadequate for treatment when administered alone. Some drugs may require administration of high doses, or more frequent administration, due to extensive metabolism into inactive or less potent metabolites. The combination therapies described herein can solve the problems mentioned above and can have one or more advantages of, e.g., synergism, reducing the number of daily doses without compromising efficacy, lowering lipids (both cholesterol and triglycerides) in patients with PBC whose elevated lipid levels are resistant to conventional therapy, improved potency, selectivity, tissue penetration, half-life, and/or metabolic stability.

In one embodiment, the disease or condition is a cholestatic liver disease. In one embodiment, the disease or condition is PBC. In another embodiment, the disease or condition is a cardiovascular disease. In another embodiment, the cardiovascular disease is atherosclerosis, hypercholesteremia, or hypertriglyceridemia.

In one aspect, the present disclosure also relates to a method of mitigating adverse events elicited or caused by OCA monotherapy (e.g., pruritus), comprising administering the disclosed combination of the compound of formula A (e.g., OCA) and a fibrate (e.g., BZF).

In another aspect, the present disclosure also provides a method for decreasing liver enzymes, comprising administering a therapeutically effective amount of the composition of the present disclosure to a subject in need thereof In one embodiment, the subject is not suffering from a cholestatic condition. In another embodiment, the subject is suffering from a cholestatic condition. In one embodiment, the liver enzyme is alkaline phosphatase, 7-glutamyl transpeptidase (GGT), and/or 5′ nucleotidase.

In certain instances, the methods described herein also include assessing, monitoring, measuring, or otherwise detecting liver function. Assessing, monitoring, measuring, or otherwise detecting liver function can be performed before, during, or after a titration period described herein, or in other instances, performed during the course of any treatment described herein. Liver function can be determined by, for example, assessing, monitoring, measuring, or otherwise detecting a level of one or more liver biomarkers compared to a control. In certain instances, the control is a baseline taken from the patient before beginning treatment. In other instances, the control is a preestablished baseline considered as a normal value. Values for measure or detection of liver function biomarkers and controls can be expressed as a comparison to Upper Limit of Normal (ULN).

In one embodiment, the methods of the present disclosure comprise a step of assessing, monitoring, measuring, or otherwise detecting liver function. In one embodiment, the step of assessing, monitoring, measuring, or otherwise detecting liver function comprises a non-invasive assay. In one embodiment, the non-invasive assay is a HepQuant SHUNT assay.

In one embodiment, the HepQuant SHUNT assay comprises measuring clearance of cholate from both the systemic circulation and portal circulation. In one embodiment, the cholate is labeled. In one embodiment, the cholate is isotopically labeled. In one embodiment, the cholate is isotopically labeled with a carbon isotope or a hydrogen isotope. In one embodiment, the cholate is isotopically labeled with ¹³C or deuterium. In one embodiment, the HepQuant SHUNT assay comprises intravenously administering (e.g., injecting) ¹³C labeled cholate. In one embodiment, the HepQuant SHUNT assay comprises orally administering deuterium labeled cholate. In one embodiment, the HepQuant SHUNT assay comprises intravenously administering ¹³C labeled cholate, and orally administering deuterium labeled cholate. In one embodiment, the HepQuant SHUNT assay comprises collecting a blood sample from the subject before the subject is administered with the cholate. In one embodiment, the HepQuant SHUNT assay comprises collecting a blood sample from the subject after cholate has been administered to the subject. In one embodiment, the HepQuant SHUNT assay comprises taking a blood sample from the subject 5, 20, 45, 60, and/or 90 minutes after administration of the cholate. In one embodiment, the HepQuant SHUNT assay comprises analyzing the blood samples to generate a Disease Severity Index (Index).

In one embodiment, the HepQuant SHUNT assay comprises:
(a) collecting a blood sample from a subject (e.g., a patient in need of treatment with the compositions, combinations, or uses described herein) before the subject is administered with cholate;
(b) intravenously administering ¹³C labeled cholate, and orally administering deuterium labeled cholate, to the subject;
(c) collecting a blood sample from the subject; and
(d) analyzing the blood samples from Steps (a) and (c) to generate a Disease Severity Index.

Liver biomarkers can be used to ascertain and quantify the efficacy of the course of treatment with the composition of the present disclosure. In other instances, liver biomarkers described herein can be used to ascertain and quantify liver function during the course of treatment with the composition of the present disclosure. Liver biomarkers can also be used to predict whether a patient or patient population is susceptible to treatment with the composition described herein. In one embodiment, the liver biomarkers include assessing, monitoring, measuring or otherwise detecting an amount or level of aspartate transaminase (AST), alanine transaminase (ALT), alkaline phosphatase (ALP), bilirubin, glycine conjugated obeticholic acid, taurine conjugated obeticholic acid, a bile acid, a bile acid glycine conjugate, or a bile acid taurine conjugate. For example, the liver biomarker assessed, monitored, measured, or detected can be ALP.

The ALP level can be a measure of ULN. In one embodiment, a patient before treatment can have an ALP level of at least 1.1×ULN to at least 20×ULN; at least 1.1×ULN to at least 15×ULN; at least 1.1×ULN to at least 12×ULN; at least 1.1×ULN to at least 10×ULN; at least 1.1×ULN to at least 8×ULN; at least 1.1×ULN to at least 6×ULN; at least 1.1×ULN to at least 5×ULN; at least 1.1×ULN to at least 4×ULN; at least 1.1×ULN to at least 3×ULN; or at least 1.1×ULN to at least 2×ULN.

A patient before a treatment described herein can have an ALP level of about 1.5×ULN to about 20×ULN; about 1.5×ULN to about 15×ULN; about 1.5×ULN to about 10ULN; about 1.5×ULN to about 5×ULN; or about 1.5×ULN to about 3×ULN. A patient before treatment can have an ALP level before a treatment described herein of about 1.5×, 2×, 3×, 4×, 5×, 8×, 10×, 15×, or 20×ULN.

A patient before a treatment described herein can have an ALP level of greater than about 1.5×, 2×, 3×, 4×, 5×, 8×, 10×, 15×, or 20×ULN. In one embodiment, a patient has an ALP level of about 1.5×ULN. In one embodiment, a patient has an ALP level of about 2×ULN. In one embodiment, a patient has a ALP level of about 5×ULN. In one embodiment, a patient has an ALP level of about 10×ULN. In one embodiment, a patient has a bilirubin level of about 15×ULN. In one embodiment, a patient has an ALP level greater than about 1.5×ULN. In one embodiment, a patient has an ALP level greater than about 2×ULN. In one embodiment, a patient has a ALP level greater than about 5×ULN. In one embodiment, a patient has an ALP level greater than about 10×ULN. In one embodiment, a patient has a bilirubin level greater than about 15×ULN.

In another example, the liver biomarker assessed, monitored, measured, or detected can be bilirubin. The bilirubin level can be a measure of ULN. In one embodiment, a patient before treatment can have a bilirubin level of at least 1.1×ULN to at least 20×ULN; at least 1.1×ULN to at least 15×ULN; at least 1.1×ULN to at least 12×ULN; at least 1.1×ULN to at least 10×ULN; at least 1.1×ULN to at least 8×ULN; at least 1.1×ULN to at least 6×ULN; at least 1.1×ULN to at least 5×ULN; at least 1.1×ULN to at least 4×ULN; at least 1.1×ULN to at least 3×ULN; or at least 1.1×ULN to at least 2×ULN.

A patient before a treatment described herein can have a bilirubin level of about 1.5×ULN to about 20×ULN; about 1.5×ULN to about 15×ULN; about 1.5×ULN to about 10 ULN; about 1.5×ULN to about 5×ULN; or about 1.5×ULN to about 3×ULN. In another example a patient before a treatment described herein can have a bilirubin level of about 2×ULN to about 20×ULN; about 2×ULN to about 15×ULN; about 2×ULN to about 10 ULN; about 2×ULN to about 5×ULN; or about 2×ULN to about 3×ULN. In another example a patient before a treatment described herein can have a bilirubin level of greater than about 2×ULN to greater than about 20×ULN; greater than about 2×ULN to greater than about 15×ULN; greater than about 2×ULN to greater than about 10 ULN; greater than about 2×ULN to greater than about 5×ULN; or greater than about 2×ULN to greater than about 3×ULN.

A patient before a treatment described herein can have a bilirubin level of about 1.5×, 2×, 3×, 4×, 5×, 8×, 10×, 15×, or 20×ULN. A patient before treatment can have a bilirubin level before a treatment described herein of greater than about 1.5×, 2×, 3×, 4×, 5×, 8×, 10×, 15×, or 20× ULN. In one embodiment, a patient has a bilirubin level greater than about 2×ULN. In one embodiment, a patient has a bilirubin level greater than about 5×ULN. In one embodiment, a patient has a bilirubin level greater than about 10×ULN. In one embodiment, a patient has a bilirubin level greater than about 15×ULN. In one embodiment, a patient has a bilirubin level less than about 2×ULN. In one embodiment, a patient has a bilirubin level less than about 5×ULN. In one embodiment, a patient has a bilirubin level less than about 10×ULN. In one embodiment, a patient has a bilirubin level less than about 15×ULN.

In some instances, it can be useful to assess, monitor, measure, or detect ALP and bilirubin to assess, monitor, measure, or otherwise detect liver function or changes in liver function during treatment with the composition described herein. In certain instances, a patient has an ALP level as provided above (e.g., about 1.5×ULN to about 10×ULN) and a bilirubin level as provided above (e.g., less than about 5×ULN). In one embodiment, the patient has an ALP level between about 1.5×ULN to about 10×ULN and a bilirubin level less than about 2×ULN.

Treatment with the composition described herein can reduce the levels of ALP and/or bilirubin in a patient described herein. For example, treatment of a disease or condition described herein (e.g., PBC) with the composition described herein can reduce the level of ALP by 2, 4, 5, 6, 8, 9, 10, 12, 15, 18, 20, 21, 22, 23, 24, 25, 26, 27, 28, 29, 30, 35, 40, 45, 50, 55, 60, 65, 70, 75, 80, 85, 88, 90, 92, 94, 96, 97, 98, 99, 99.2, 99.4, 99.6, 99.7, 99.8, 99.9, or 100%. In another example, the level of ALP can be reduced by at least 100%, at least 125%, at least 150%, at least 175%, at least 200%, at least 225%, at least 250% or at least 300%.

In another example, the level of ALP can be reduced by about 5% to about 50%; about 10% to about 55%; about 10% to about 45%; about 10% to about 40%; about 10% to about 33%, about 10% to about 30%; about 15% to about 30%; about 15% to about 25%; about 20% to about 50%, about 20% to about 40%; about 20% to about 35%; about 20% to about 30%; 20% to about 27%; or about 20% to about 27%. In another example, the level of ALP can be reduced by at least 50%. The level of ALP can be reduced by at least 40%. The level of ALP can be reduced by at least 35%. The level of ALP can be reduced by at least 30%. The level of ALP can be reduced by at least 27%. The level of ALP can be reduced by at least 25%. The level of ALP can be reduced by at least 20%.

The reduction of ALP levels can be represented by the fold change over ULN. For example, treatment with the composition described herein can reduce the ALP level of a patient described herein to less than about 5×ULN; less than about 4×ULN, less than about 3×ULN, less than about 2×ULN, less than about 1.7×ULN, less than about 1.5×ULN, less than about 1.25×ULN, or less than about ULN.

In another example, the ALP level is reduced by at least 1, 2, 3, 4, 5, 6, 7, 8, 9, 10, 15, 20, 25, 30, 40, or 5-fold compared to a baseline value. For example, the ALP level after treatment with the composition described herein can be reduced by 1, 1.2, 1.4, 1.6, 1.8, or 2-fold, including intervening values therein, compared to a baseline value. In another example, the ALP level can be reduced by 2, 2.2, 2.4, 2.6, 2.8, or 3-fold, including intervening values therein, compared to a baseline value. In another example, the ALP level can be reduced 3, 4, or 5-fold, including intervening values therein, compared to a baseline value. In another example, the ALP level can be reduced 5, 7, 9, or 10-fold, including intervening values therein, compared to a baseline value. In another example, the ALP level can be reduced 10, 12, 15, or 20-fold, including intervening values therein, compared to a baseline value.

Treatment of a disease or condition described herein (e.g., PBC) with the composition described herein can reduce the level of bilirubin by 2, 4, 5, 6, 8, 9, 10, 12, 15, 18, 20, 21, 22, 23, 24, 25, 26, 27, 28, 29, 30, 35, 40, 45, 50, 55, 60, 65, 70, 75, 80, 85, 88, 90, 92, 94, 96, 97, 98, 99, 99.2, 99.4, 99.6, 99.7, 99.8, 99.9, or 100%. In another example, the level of bilirubin can be reduced by at least 100%, at least 125%, at least 150%, at least 175%, at least 200%, at least 225%, at least 250% or at least 300%.

In another example, the level of bilirubin can be reduced by about 5% to about 50%; about 10% to about 55%; about 10% to about 45%; about 10% to about 40%; about 10% to about 33%, about 10% to about 30%; about 15% to about 30%; about 15% to about 25%; about 20% to about 50%, about 20% to about 40%; about 20% to about 35%; about 20% to about 30%; 20% to about 27%; or about 20% to about 27%. In another example, the level of bilirubin can be reduced by at least 50%. The level of bilirubin can be reduced by at least 40%. The level of bilirubin can be reduced by at least 35%. The level of bilirubin can be reduced by at least 30%. The level of bilirubin can be reduced by at least 27%. The level of bilirubin can be reduced by at least 25%. The level of bilirubin can be reduced by at least 20%.

The reduction of bilirubin levels can be represented by the fold change over ULN. For example, treatment with the composition described herein can reduce the bilirubin level of a patient described herein to less than about 5×ULN; less than about 4×ULN, less than about 3×ULN, less than about 2×ULN, less than about 1.7×ULN, less than about 1.5×ULN, less than about 1.25×ULN, or less than about ULN.

In another example, the bilirubin level is reduced by at least 1, 2, 3, 4, 5, 6, 7, 8, 9, 10, 15, 20, 25, 30, 40, or 50-fold compared to a baseline value. For example, the bilirubin level after treatment with the composition described herein can be reduced by 1, 1.2, 1.4, 1.6, 1.8, or 2-fold, including intervening values therein, compared to a baseline value. In another example, the bilirubin level can be reduced by 2, 2.2, 2.4, 2.6, 2.8, or 3-fold, including intervening values therein, compared to a baseline value. In another example, the bilirubin level can be reduced 3, 4, or 5-fold, including intervening values therein, compared to a baseline value. In another example, the bilirubin level can be reduced 5, 7, 9, or 10-fold, including intervening values therein, compared to a baseline value. In another example, the bilirubin level can be reduced 10, 12, 15, or 20-fold, including intervening values therein, compared to a baseline value.

In another embodiment, one or more biomarkers can stratify a patient population undergoing undergo treatment with the composition described herein. For example, a PBC patient can be stratified for the risk of hepatocellular carcinoma (HCC).

In another embodiment, liver biomarkers useful for detection can include metabolites and bile acids. For example, assessing, monitoring, measuring, or otherwise detecting levels of glycine and taurine conjugates of compounds of formula A (e.g., obeticholic acid) can be useful for measuring efficacy of a treatment regimen described herein. For example, assessing, monitoring, measuring, or otherwise detecting levels or detecting plasma levels of bile acids including cholic acid, chenodeoxycholic acid, deoxycholic acid, lithocholic acid, and ursodeoxycholic acid, including glycine and taurine conjugates thereof, and optionally comparing the levels to a control, can be useful for measuring efficacy of a treatment regimen described herein.

In still other embodiments, calculating an AST to platelet index (APRI) can be useful for assessing, monitoring, measuring, or otherwise detecting liver function (including changes therein). The compositions described herein can reduce the APRI of a patient described herein. In certain instances, monitoring or measuring the APRI can be used to determine efficacy of treatment with the composition described herein. In some embodiments, a reduction in APRI is observed in a patient (e.g., a PBC patient) after administration of the composition described herein. For example, the APRI may be reduced by about 5% to about 50% in patients treated with the composition of the present disclosure relative to baseline levels measured before dose administration. The reduction may be up to about 5%, 10%, 15%, 20%, 25%, 30%, 35%, 40%, 45% or 50%.

The present disclosure relates to a method for treating primary biliary cirrhosis (PBC) in a patient in need thereof, the method comprising: (1) administering to the patient a composition comprising a compound of formula A (e.g., OCA) and a fibrate (e.g., BZF); (2) assessing liver function (optionally before, during, and after said titration period) of the patient by: (a) calculating an AST to platelet ratio (APRI) score for the patient; (b) measuring the level of one or more liver biomarkers selected from ALP, bilirubin, AST, ALT, glycine conjugated obeticholic acid, taurine conjugated obeticholic acid, a bile acid, a bile acid glycine conjugate, or a bile acid taurine conjugate; or (c) a HepQuant SHUNT assay described herein; (3) wherein a reduced APRI score compared to a control or a reduced level of the one or more liver biomarkers compared to a control indicates non-impaired liver function; (4) assessing tolerance of the patient to the starting dose by grading the severity of one or more adverse effects, if present; and (5) administering an adjusted dose of the composition (if necessary) (wherein the adjusted dose comprises an amount equal to or greater than an amount of the starting dose).

The present disclosure relates to a composition comprising a compound of formula A (e.g., OCA) or a pharmaceutically acceptable salt, ester, or amino acid conjugate thereof and a fibrate (e.g., BZF) for use in treating primary biliary cirrhosis (PBC) in a patient in need thereof wherein the composition is prepared to be administered (optionally in a titration period) wherein
the liver function of the patient is assessed (optionally before, during, and after said titration period) by calculating an AST to platelet ratio (APRI) score for said patient or by measuring the level of one or more liver biomarkers selected from ALP, bilirubin, AST, ALT, glycine conjugated obeticholic acid, taurine conjugated obeticholic acid, a bile acid, a bile acid glycine conjugate, or a bile acid taurine conjugate, wherein a reduced APRI score compared to a control or a reduced level of said one or more liver biomarkers compared to a control indicates non-impaired liver function; and
the tolerance of the patient to said starting dose is assessed by grading the severity of one or more adverse effects, if present; and the composition is prepared to be administered as an adjusted dose (wherein said adjusted dose comprises an amount equal to or greater than an amount of said starting dose).

The present disclosure relates to a method for treating primary biliary cirrhosis (PBC) in a patient in need thereof, the method comprising: (1) administering the composition comprising OCA or a pharmaceutically acceptable salt or amino acid conjugate thereof in the amount of 5-50 mg once daily (QD) and bezafibrate in the amount of 200-400 mg once daily (QD); (2) assessing liver function (optionally before, during, and after said titration period) of the patient by: (a) calculating an AST to platelet ratio (APRI) score for the patient; (b) measuring the level of one or more liver biomarker selected from ALP, bilirubin, AST, ALT, glycine conjugated obeticholic acid, taurine conjugated obeticholic acid, a bile acid, a bile acid glycine conjugate, or a bile acid taurine conjugate; or (c) a HepQuant SHUNT assay described herein; (3) wherein a reduced APRI score compared to a control or a reduced level of the one or more liver biomarkers compared to a control indicates non-impaired liver function; (4) assessing tolerance of the patient to the starting dose by grading the severity of one or more adverse effects, if present; and (5) administering an adjusted dose of the composition (if necessary) (wherein the adjusted dose comprises an amount equal to or greater than an amount of the starting dose).

The present disclosure relates to a method for treating PBC in a patient in need thereof, the method comprising administering a composition comprising OCA or a pharmaceutically acceptable salt or amino acid conjugate thereof in the amount of 5-50 mg and bezafibrate in the amount of 200-400 mg, wherein the composition is administered once daily (QD).

The present disclosure relates to a method for treating PBC in a patient in need thereof, the method comprising administering a composition comprising OCA or a pharmaceutically acceptable salt or amino acid conjugate thereof in the amount of 5 mg and bezafibrate in the amount of 200 mg, wherein the composition is administered QD.

The present disclosure relates to a method for treating PBC in a patient in need thereof, the method comprising administering a composition comprising OCA or a pharmaceutically acceptable salt or amino acid conjugate thereof in the amount of 5 mg and bezafibrate in the amount of 400 mg, wherein the composition is administered QD.

The present disclosure relates to a method for treating PBC in a patient in need thereof, the method comprising administering a composition comprising OCA or a pharmaceutically acceptable salt or amino acid conjugate thereof in the amount of 10 mg and bezafibrate in the amount of 200 mg, wherein the composition is administered QD.

The present disclosure relates to a method for treating PBC in a patient in need thereof, the method comprising administering a composition comprising OCA or a pharmaceutically acceptable salt or amino acid conjugate thereof in the amount of 10 mg and bezafibrate in the amount of 400 mg, wherein the composition is administered QD.

The present disclosure relates to a composition comprising OCA or a pharmaceutically acceptable salt or amino acid conjugate thereof in the amount of 5-50 mg and bezafibrate in the amount of 200-400 mg for use in the treatment of PBC, wherein the composition is for administration once daily.

The present disclosure relates to a composition comprising OCA or a pharmaceutically acceptable salt or amino acid conjugate thereof in the amount of 5 mg and bezafibrate in the amount of 200 mg for use in the treatment of PBC, wherein the composition is for administration once daily.

The present disclosure relates to a composition comprising OCA or a pharmaceutically acceptable salt or amino acid conjugate thereof in the amount of 5 mg and bezafibrate in the amount of 400 mg for use in the treatment of PBC, wherein the composition is for administration once daily.

The present disclosure relates to a composition comprising OCA or a pharmaceutically acceptable salt or amino acid conjugate thereof in the amount of 10 mg and bezafibrate in the amount of 200 mg for use in the treatment of PBC, wherein the composition is for administration once daily.

The present disclosure relates to a composition comprising OCA or a pharmaceutically acceptable salt or amino acid conjugate thereof in the amount of 10 mg and bezafibrate in the amount of 400 mg for use in the treatment of PBC, wherein the composition is for administration once daily.

The present disclosure relates to use of a composition comprising OCA or a pharmaceutically acceptable salt or amino acid conjugate thereof in the amount of 5-50 mg and bezafibrate in the amount of 200-400 mg in the manufacture of a medicament for the treatment of PBC, wherein the composition is for administration once daily.

The present disclosure relates to use of a composition comprising OCA or a pharmaceutically acceptable salt or amino acid conjugate thereof in the amount of 5 mg and bezafibrate in the amount of 200 mg in the manufacture of a medicament for the treatment of PBC, wherein the composition is for administration once daily.

The present disclosure relates to use of a composition comprising OCA or a pharmaceutically acceptable salt or amino acid conjugate thereof in the amount of 5 mg and bezafibrate in the amount of 400 mg in the manufacture of a medicament for the treatment of PBC, wherein the composition is for administration once daily.

The present disclosure relates to use of a composition comprising OCA or a pharmaceutically acceptable salt or amino acid conjugate thereof in the amount of 10 mg and bezafibrate in the amount of 200 mg in the manufacture of a medicament for the treatment of PBC, wherein the composition is for administration once daily.

The present disclosure relates to use of a composition comprising OCA or a pharmaceutically acceptable salt or amino acid conjugate thereof in the amount of 10 mg and bezafibrate in the amount of 400 mg in the manufacture of a medicament for the treatment of PBC, wherein the composition is for administration once daily.

The present disclosure relates to a method for treating PBC in a patient in need thereof, the method comprising administering to the patient OCA or a pharmaceutically acceptable salt or amino acid conjugate thereof in the amount of 5-50 mg QD and bezafibrate in the amount of 200-400 mg QD.

The present disclosure relates to a method for treating PBC in a patient in need thereof, the method comprising administering to the patient OCA or a pharmaceutically acceptable salt or amino acid conjugate thereof in the amount of 5 mg QD and bezafibrate in the amount of 200 mg QD.

The present disclosure relates to a method for treating PBC in a patient in need thereof, the method comprising administering to the patient OCA or a pharmaceutically acceptable salt or amino acid conjugate thereof in the amount of 5 mg QD and bezafibrate in the amount of 400 mg QD.

The present disclosure relates to a method for treating PBC in a patient in need thereof, the method comprising administering to the patient OCA or a pharmaceutically acceptable salt or amino acid conjugate thereof in the amount of 10 mg QD and bezafibrate in the amount of 200 mg QD.

The present disclosure relates to a method for treating PBC in a patient in need thereof, the method comprising administering to the patient OCA or a pharmaceutically acceptable salt or amino acid conjugate thereof in the amount of 10 mg QD and bezafibrate in the amount of 400 mg QD.

The present disclosure relates to OCA or a pharmaceutically acceptable salt or amino acid conjugate thereof for use in combination with bezafibrate in the treatment of PBC, wherein OCA or a pharmaceutically acceptable salt or amino acid conjugate thereof is for administration in the amount of 5-50 mg QD and bezafibrate is for administration in the amount of 200-400 mg QD.

The present disclosure relates to OCA or a pharmaceutically acceptable salt or amino acid conjugate thereof for use in combination with bezafibrate in the treatment of PBC, wherein OCA or a pharmaceutically acceptable salt or amino acid conjugate thereof is for administration in the amount of 5 mg QD and bezafibrate is for administration in the amount of 200 mg QD.

The present disclosure relates to OCA or a pharmaceutically acceptable salt or amino acid conjugate thereof for use in combination with bezafibrate in the treatment of PBC, wherein OCA or a pharmaceutically acceptable salt or amino acid conjugate thereof is for administration in the amount of 5 mg QD and bezafibrate is for administration in the amount of 400 mg QD.

The present disclosure relates to OCA or a pharmaceutically acceptable salt or amino acid conjugate thereof for use in combination with bezafibrate in the treatment of PBC, wherein OCA or a pharmaceutically acceptable salt or amino acid conjugate thereof is for administration in the amount of 10 mg QD and bezafibrate is for administration in the amount of 200 mg QD.

The present disclosure relates to OCA or a pharmaceutically acceptable salt or amino acid conjugate thereof for use in combination with bezafibrate in the treatment of PBC, wherein OCA or a pharmaceutically acceptable salt or amino acid conjugate thereof is for administration in the amount of 10 mg QD and bezafibrate is for administration in the amount of 400 mg QD.

The present disclosure relates to use of OCA or a pharmaceutically acceptable salt or amino acid conjugate thereof in combination with bezafibrate in the manufacture of a medicament for use in the treatment of PBC, wherein OCA or a pharmaceutically acceptable salt or amino acid conjugate thereof is for administration in the amount of 5-50 mg QD and bezafibrate is for administration in the amount of 200-400 mg QD.

The present disclosure relates to use of OCA or a pharmaceutically acceptable salt or amino acid conjugate thereof in combination with bezafibrate in the manufacture of a medicament for use in the treatment of PBC, wherein OCA or a pharmaceutically acceptable salt or amino acid conjugate thereof is for administration in the amount of 5 mg QD and bezafibrate is for administration in the amount of 200 mg QD.

The present disclosure relates to use of OCA or a pharmaceutically acceptable salt or amino acid conjugate thereof in combination with bezafibrate in the manufacture of a medicament for use in the treatment of PBC, wherein OCA or a pharmaceutically acceptable salt or amino acid conjugate thereof is for administration in the amount of 5 mg QD and bezafibrate is for administration in the amount of 400 mg QD.

The present disclosure relates to use of OCA or a pharmaceutically acceptable salt or amino acid conjugate thereof in combination with bezafibrate in the manufacture of a medicament for use in the treatment of PBC, wherein OCA or a pharmaceutically acceptable salt or amino acid conjugate thereof is for administration in the amount of 10 mg QD and bezafibrate is for administration in the amount of 200 mg QD.

The present disclosure relates to use of OCA or a pharmaceutically acceptable salt or amino acid conjugate thereof in combination with bezafibrate in the manufacture of a medicament for use in the treatment of PBC, wherein OCA or a pharmaceutically acceptable salt or amino acid conjugate thereof is for administration in the amount of 10 mg QD and bezafibrate is for administration in the amount of 400 mg QD.

The present disclosure relates to a combinational therapy for the treatment of PBC, comprising administration of OCA or a pharmaceutically acceptable salt or amino acid conjugate thereof in the amount of 5-50 mg QD and bezafibrate in the amount of 200-400 mg QD.

The present disclosure relates to a combinational therapy for the treatment of PBC, comprising administration of OCA or a pharmaceutically acceptable salt or amino acid conjugate thereof in the amount of 5 mg QD and bezafibrate in the amount of 200 mg QD.

The present disclosure relates to a combinational therapy for the treatment of PBC, comprising administration of OCA or a pharmaceutically acceptable salt or amino acid conjugate thereof in the amount of 5 mg QD and bezafibrate in the amount of 400 mg QD.

The present disclosure relates to a combinational therapy for the treatment of PBC, comprising administration of OCA or a pharmaceutically acceptable salt or amino acid conjugate thereof in the amount of 10 mg QD and bezafibrate in the amount of 400 mg QD.

The present disclosure relates to a combinational therapy for the treatment of PBC, comprising administration of OCA or a pharmaceutically acceptable salt or amino acid conjugate thereof in the amount of 10 mg QD and bezafibrate in the amount of 400 mg QD.

In one embodiment, the methods, combinations for use, uses, and combination therapies of the present application comprise administering or administration for a period of at least 4 weeks. In one embodiment, the methods, combinations for use, uses, and combination therapies of the present application comprise administering or administration for a period of at least 12 weeks. In one embodiment, the methods, combinations for use, uses, and combination therapies of the present application comprise administering or administration for a period of 1-12 weeks. In one embodiment, the methods, combinations for use, uses, and combination therapies of the present application comprise administering or administration for a period of 12-48 weeks.

In one embodiment, OCA or a pharmaceutically acceptable salt or amino acid conjugate thereof is in a tablet form.

In one embodiment, bezafibrate is in an immediate release form (e.g., immediate release tablet). In one embodiment, bezafibrate is in a sustained release form (e.g., sustained release tablet).

In one embodiment, the methods, combinations for use, uses, and combination therapies of the present application further comprise a step of assessing, monitoring, measuring, or otherwise detecting liver function, as described herein (e.g., HepQuant SHUNT assay).

Further provided herein is a method for treating PBC in a patient in need thereof by administering a starting dose of a composition (or an FXR agonist, e.g., a compound of formula A) described herein in a titration period. The method includes assessing liver function of the patient before, during, and after said titration period by either calculating an APRI score for said patient; or by measuring the level of one or more liver biomarkers selected from ALP, bilirubin, AST, ALT, glycine conjugated obeticholic acid, taurine conjugated obeticholic acid, a bile acid, a bile acid glycine conjugate, or a bile acid taurine conjugate, where a reduced APRI score compared to a control or a reduced level of the one or more liver biomarkers compared to a control indicates non-impaired liver function. The method further includes assessing tolerance of the patient to the starting dose by grading the severity of one or more adverse effects, if present, and administering an adjusted dose of the composition (or adjusted dose of the compound of formula A, e.g., OCA), where the adjusted dose includes an amount equal to or greater than an amount of the starting dose. The starting dose, adjusted dose, and titration period are as described below. For example, the starting dose can be about 5 to about 50 mg (e.g., 5 mg) and the adjusted dose can be about 5 to about 50 mg (e.g., 5 mg, 10 mg, or 25 mg) and the titration period can be a time of about 1 to about 6 months, e.g., 1 month, 2 months, 3 months, 4 months, 5 months, or 6 months.

Also provided herein are methods to reduce or eliminate rejection failure of a liver transplant by administering an effective amount of the composition described herein. In certain instances, administration of the composition described herein reduces expression or levels of ALP and/or bilirubin. In one embodiment, administration of the composition described herein reduces ALP and bilirubin levels, thereby reducing transplant complications or rejection. In another embodiment, administration of an effective amount of the composition described herein increases post-transplantation survival rate of a liver transplantee.

In the compositions, packs or kits, methods and uses of the present invention, the compound of formula A may be in a free form (e.g., acid) or it may be a pharmaceutically acceptable salt or amino acid conjugate (e.g., glycine or taurine conjugate) thereof. In one aspect, the compound is any FXR agonist. In one aspect, the compound is a compound of formula A. In one aspect, the compound of formula A is a compound of formula 1 (obeticholic acid or OCA). In one aspect, the compound of formula A is a compound of formula 2. In one aspect, the compound of formula A is a compound of formula 3. In one aspect, the compound of formula A is the pharmaceutically acceptable salt of a compound of formula 3. In one aspect, the compound of formula A is a compound of formula 3a or 3b.

In the compositions, packs or kits, methods and uses of the present invention, the fibrate can be any fibrate. In one aspect, the fibrate is selected from the group consisting of fenofibrate, bezafibrate, beclobrate, binifibrate, ciprofibrate, clinofibrate, clofibrate, clofibric acid, etofibrate, gemfibrozil, nicofibrate, pirifibrate, ronifibrate, simfibrate, theofibrate, tocofibrate, plafibride, and a pharmaceutically acceptable salt and ester thereof, and derivatives of 2-phenoxy-2-methylpropanoic acid in which the phenoxy moiety is substituted with an optionally substituted residue of piperidine, 4-hydroxypiperidine, piperid-3-ene or piperazine, as disclosed in European Patent Application Publication No. EP0607536. In one aspect, the fibrate is selected from the group consisting of bezafibrate, ciprofibrate, clofibrate, fenofibrate, gemfibrozil, binifibrate, clinofibrate, clofibric acid, nicofibrate, pirifibrate, plafibride, ronifibrate, theofibrate, tocofibrate, and a pharmaceutically acceptable salt and ester thereof, and derivatives of 2-phenoxy-2-methylpropanoic acid, in which the phenoxy moiety is substituted with an optionally substituted residue of piperidine, 4-hydroxypiperidine, piperid-3-ene or piperazine, as disclosed in European Patent Application Publication No. EP0607536. An example of the latter group of substances is 2-[3-[1-(4-fluorobenzoyl)piperidin-4-yl]phenoxy-2-methyl-propanoic acid. For example, the fibrate is bezafibrate, fenofibrate, gemfibrozil, ciprofibrate, clofibrate, clofibric acid, or a pharmaceutically acceptable salt or ester thereof. In one embodiment, the fibrate is bezafibrate (BZF).

In one embodiment, the compound of formula A is the free form (e.g., acid) of a compound of formula A, and the at least one fibrate is selected from bezafibrate, fenofibrate, gemfibrozil, ciprofibrate, clofibrate, and a pharmaceutically acceptable salt or ester thereof.

In one embodiment, the compound of formula A is a pharmaceutically acceptable salt of compound of formula A, and the at least one fibrate is selected from bezafibrate, fenofibrate, gemfibrozil, ciprofibrate, clofibrate, and a pharmaceutically acceptable salt or ester thereof.

In one embodiment, the compound of formula A is the glycine conjugate of a compound of formula A, and the at least one fibrate is selected from bezafibrate, fenofibrate, gemfibrozil, ciprofibrate, clofibrate, and a pharmaceutically acceptable salt or ester thereof.

In one embodiment, the compound of formula A is the taurine conjugate of a compound of formula A, and the at least one fibrate is selected from bezafibrate, fenofibrate, gemfibrozil, ciprofibrate, clofibrate, and pharmaceutically acceptable salts or esters thereof

In one embodiment, the compound of formula A is a compound of formula A (free form) or a pharmaceutically acceptable salt or amino acid conjugate, and the at least one fibrate is bezafibrate.

The invention also encompasses an isotopically-labeled compound of formula A or a pharmaceutically acceptable salt or amino acid conjugate thereof, which has a structure that is identical to that of the compound of formula A of the present invention except that one or more atoms is replaced by an atom having an atomic mass or mass number different from the atomic mass or mass number most commonly found in nature. Examples of isotopes that can be incorporated into the compound of formula A or a pharmaceutically acceptable salt or amino acid conjugate thereof, include isotopes of hydrogen, carbon, nitrogen, fluorine, such as ³H, ¹¹C, ¹⁴C and ¹⁸F.

The compound of formula A or a pharmaceutically acceptable salt or amino acid conjugate thereof that contains the aforementioned isotopes and/or other isotopes of other atoms is within the scope of the present invention. Isotopically labeled compounds of formula A or a pharmaceutically acceptable salt or amino acid conjugate thereof, for example, a compound of formula A into which a radioactive isotope(s) such as ³H and/or ¹⁴C are incorporated, are useful in drug and/or substrate tissue distribution assays. Tritiated, i.e., ³H, and carbon-14, i.e., ¹⁴C, isotopes are used for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be used in some circumstances. Isotopically labeled compounds of formula A or a pharmaceutically acceptable salt or amino acid conjugate thereof can be prepared by carrying out the procedures disclosed in the Schemes and/or in the Examples of the present disclosure, and substituting a readily available isotopically labeled reagent for a non-isotopically labeled reagent.

The present invention also provides a method for treating or preventing a disease or condition, comprising administering a therapeutically effective amount of a pharmaceutical composition of the present invention to a subject in need thereof.

In one embodiment, the disease or condition is an FXR mediated disease or condition. Examples of the FXR mediated diseases or conditions include, but are not limited to, liver diseases (including cholestatic liver diseases) such as, for example, primary biliary cholangitis (PBC), primary sclerosing cholangitis (PSC), and biliary atresia. In one embodiment, the disease or condition is a cholestatic liver disease. In one embodiment, the disease or condition is PBC.

The present invention also provides a method of mitigating adverse events elicited or caused by OCA monotherapy (e.g., pruritus), comprising administering the disclosed combination of the compound of formula A (e.g., OCA) and a fibrate (e.g., BZF).

The present invention also provides a method for inhibiting or reversing fibrosis associated with a disease or condition described herein, comprising administering a therapeutically effective amount of a pharmaceutical composition of the present invention to a subject in need thereof. In another embodiment, the subject is suffering from a cholestatic condition. In embodiments, the fibrosis to be inhibited or reversed occurs in an organ where FXR is expressed.

In one embodiment, a cholestatic condition is defined as having an abnormally elevated serum level of alkaline phosphatase, y-glutamyl transpeptidase (GGT), and/or 5′ nucleotidase. In another embodiment, a cholestatic condition is further defined as presenting with at least one clinical symptom. In one embodiment, the symptom is itching (pruritus). In another embodiment, a cholestatic condition is selected from the group consisting of primary biliary cholangitis (PBC), primary sclerosing cholangitis (PSC), drug-induced cholestasis, hereditary cholestasis, biliary atresia, and intrahepatic cholestasis of pregnancy.

The present invention also provides a method for reducing lipid levels (i.e., amount of lipid), such as in the blood, comprising administering a therapeutically effective amount of a pharmaceutical composition of the present invention to a subject in need thereof. In one embodiment, the method of the present invention reduces the lipid levels by at least 10%, 20%, 30%, 40%, 50%, 60%, 70%, 80%, or 90%, as compared to a control subject (e.g., a subject not administered with the composition of the present invention). In one embodiment, the subject has elevated levels of lipid, as compared to a healthy subject (e.g., an individual without a disease or condition, such as those described herein). In one embodiment, the method of the present application reduces the levels of lipid to normal levels (e.g., similar to the lipid levels in an individual without a disease or condition, such as those described herein).

In one embodiment, the lipid is cholesterol. In one embodiment, the method of the present invention reduces cholesterol levels by at least 10%, 20%, 30%, 40%, 50%, 60%, 70%, 80%, or 90%, as compared to a control subject (e.g., a subject not administered with the composition of the present invention). In one embodiment, the subject has elevated levels of cholesterol, as compared to a healthy subject (e.g., an individual without a disease or condition, such as those described herein). In one embodiment, the method of the present invention reduces cholesterol levels below 400 mg/L, 350 mg/L, 300 mg/L, 250 mg/L, 240 mg/L, 230 mg/L, 220 mg/L, 210 mg/L, 200 mg/L, 190 mg/L, 180 mg/L, 170 mg/L, 160 mg/L, or 150 mg/L. In one embodiment, the method of the present invention reduces cholesterol levels below 200 mg/L, 190 mg/L, 180 mg/L, 170 mg/L, 160 mg/L, or 150 mg/L.

In one embodiment, the cholesterol is LDL. In one embodiment, the method of the present invention reduces LDL levels by at least 10%, 20%, 30%, 40%, 50%, 60%, 70%, 80%, or 90%, as compared to a control subject (e.g., a subject not administered with the composition of the present invention). In one embodiment, the subject has elevated levels of LDL, as compared to a healthy subject (e.g., an individual without a disease or condition, such as those described herein). In one embodiment, the method of the present invention reduces LDL levels below 300 mg/L, 200 mg/L, 190 mg/L, 180 mg/L, 170 mg/L, 160 mg/L, 150 mg/L, 140 mg/L, 130 mg/L, 120 mg/L, 110 mg/L, 100 mg/L, 90 mg/L, 80 mg/L, 70 mg/L, 60 mg/L, or 50 mg/L. In one embodiment, the method of the present invention reduces LDL levels below 160 mg/L, 150 mg/L, 140 mg/L, 130 mg/L, 120 mg/L, 110 mg/L, 100 mg/L, 90 mg/L, 80 mg/L, 70 mg/L, 60 mg/L, or 50 mg/L. In one embodiment, the method of the present invention reduces LDL levels below 130 mg/L, 120 mg/L, 110 mg/L, 100 mg/L, 90 mg/L, 80 mg/L, 70 mg/L, 60 mg/L, or 50 mg/L. In one embodiment, the method of the present invention reduces LDL levels below 100 mg/L, 90 mg/L, 80 mg/L, 70 mg/L, 60 mg/L, or 50 mg/L. In one embodiment, the method of the present invention reduces LDL levels below 70 mg/L, 60 mg/L, or 50 mg/L.

In one embodiment, the lipid is triglyceride. In one embodiment, the method of the present invention reduces triglyceride levels by at least 10%, 20%, 30%, 40%, 50%, 60%, 70%, 80%, or 90%, as compared to a control subject (e.g., a subject not administered with the composition of the present invention). In one embodiment, the subject has elevated levels of triglyceride, as compared to a healthy subject (e.g., an individual without a disease or condition, such as those described herein). In one embodiment, the method of the present invention reduces triglyceride levels below 800 mg/L, 700 mg/L, 600 mg/L, 500 mg/L, 400 mg/L, 300 mg/L, 200 mg/L, 190 mg/L, 180 mg/L, 170 mg/L, 160 mg/L, 150 mg/L, 140 mg/L, 130 mg/L, 120 mg/L, 110 mg/L, or 100 mg/L. In one embodiment, the method of the present invention reduces triglyceride levels below 200 mg/L, 190 mg/L, 180 mg/L, 170 mg/L, 160 mg/L, 150 mg/L, 140 mg/L, 130 mg/L, 120 mg/L, 110 mg/L, or 100 mg/L. In one embodiment, the method of the present invention reduces triglyceride levels below 150 mg/L, 140 mg/L, 130 mg/L, 120 mg/L, 110 mg/L, or 100 mg/L.

The present invention also provides a method for reducing the amount of bilirubin, and/or one or more liver enzymes, comprising administering a therapeutically effective amount of a pharmaceutical composition of the present invention to a subject in need thereof.

In one embodiment, the method of the present application reduces the amount of bilirubin by at least 10%, 20%, 30%, 40%, 50%, 60%, 70%, 80%, or 90%, as compared to a control subject (e.g., a subject not administered with the composition of the present invention). In one embodiment, the subject has an elevated level of bilirubin, as compared to a healthy subject (e.g., an individual without a disease or condition, such as those described herein). In one embodiment, the method of the present application reduces the level of bilirubin to a normal level (e.g., similar to the level of bilirubin in an individual without a disease or condition, such as those described herein). In a further embodiment, the method of the present application reduces the level of bilirubin below 10 mg/L, 9 mg/L, 8 mg/L, 7 mg/L, 6 mg/L, 5 mg/L, 4 mg/L, 3 mg/L, 2 mg/L, 1.5 mg/L, 1.2 mg/L, or 1 mg/L. In a further embodiment, the method of the present application reduces the level of bilirubin below 2 mg/L, 1.5 mg/L, 1.2 mg/L, or 1 mg/L.

In one embodiment, the liver enzyme is selected from the group consisting of alkaline phosphatase (ALP, AP, or Alk Phos), alanine aminotransferase (ALT), aspartate aminotransferase (AST), gamma-glutamyl transpeptidase (GGT), lactate dehydrogenase (LDH), and 5′ nucleotidase. In one embodiment, the method of the present application reduces the amount of one or more liver enzymes by at least 10%, 20%, 30%, 40%, 50%, 60%, 70%, 80%, or 90%, as compared to a control subject (e.g., a subject not administered with the composition of the present invention). In one embodiment, the subject has elevated levels of one or more liver enzymes, as compared to a healthy subject (e.g., an individual without a disease or condition, such as those described herein). In one embodiment, the method of the present application reduces the levels of one or more liver enzymes (e.g., ALP, ALT, AST, GGT, LDH, and 5′ nucleotidase) to normal levels (e.g., similar to the levels of liver enzymes in an individual without a disease or condition, such as those described herein).

In a further embodiment, the method of the present application reduces the level of ALP below 500 IU/L (international units per liter), 400 IU/L, 300 IU/L, 200 IU/L, 180 IU/L, 160 IU/L, or 150 IU/L. In a further embodiment, the method of the present application reduces the level of ALP to from about 40 IU/L to about 150 IU/L.

In a further embodiment, the method of the present application reduces the level of ALT below 200 IU/L (international units per liter), 150 IU/L, 100 IU/L, 80 IU/L, 60 IU/L, or 50 IU/L. In a further embodiment, the method of the present application reduces the level of ALT to from about 5 IU/L to about 50 IU/L.

In a further embodiment, the method of the present application reduces the level of AST below 200 IU/L (international units per liter), 150 IU/L, 100 IU/L, 80 IU/L, 60 IU/L, 50 IU/L, or 40 IU/L. In a further embodiment, the method of the present application reduces the level of AST to from about 10 IU/L to about 50 IU/L.

In a further embodiment, the method of the present application reduces the level of GGT below 200 IU/L (international units per liter), 150 IU/L, 100 IU/L, 90 IU/L, 80 IU/L, 70 IU/L, or 60 IU/L. In a further embodiment, the method of the present application reduces the level of GGT to from about 15 IU/L to about 50 IU/L or from about 5 IU/L to about 30 IU/L.

In a further embodiment, the method of the present application reduces the level of LDH below 500 IU/L (international units per liter), 400 IU/L, 300 IU/L, 200 IU/L, 180 IU/L, 160 IU/L, 150 IU/L, 140 IU/L, or 130 IU/L. In a further embodiment, the method of the present application reduces the level of LDH to from about 120 IU/L to about 220 IU/L.

In a further embodiment, the method of the present application reduces the level of 5′ nucleotidase below 50 IU/L (international units per liter), 40 IU/L, 30 IU/L, 20 IU/L, 18 IU/L, 17 IU/L, 16 IU/L, 15 IU/L, 14 IU/L, 13 IU/L, 12 IU/L, 11 IU/L, 10 IU/L, 9 IU/L, 8 IU/L, 7 IU/L, 6 IU/L, or 5 IU/L. In a further embodiment, the method of the present application reduces the level of 5′ nucleotidase to from about 2 IU/L to about 15 IU/L.

In one embodiment, the methods of the present invention comprise administering to a subject in need thereof an effective amount of an FXR agonist, in combination with at least one fibrate, and optionally one or more pharmaceutically acceptable carriers. In a further embodiment, the method comprises administering to a subject in need thereof an effective amount of a compound of formula A or Compound 1, 2, or 3 (including 3 and 3b) or a pharmaceutically acceptable salt or amino acid conjugate thereof, a fibrate and optionally one or more pharmaceutically acceptable carriers.

In one embodiment, the methods of the present invention comprise administering to a subject in need thereof an effective amount of an FXR agonist, in combination with at least one fibrate, and optionally one or more pharmaceutically acceptable carriers. In a further embodiment, the method comprises administering to a subject in need thereof an effective amount of a compound of formula A or Compound 1, 2, or 3 (including 3 and 3b) or a pharmaceutically acceptable salt or amino acid conjugate thereof in combination with at least one fibrate and optionally one or more pharmaceutically acceptable carriers.

In one embodiment, the subject is a mammal. In one embodiment, the mammal is a human.

In a further embodiment, the compound of formula A and a fibrate are administered in a two-way combination, i.e., without any therapeutic agent other than the compound of formula A and a fibrate. It can be particularly advantageous for such a combination of a compound of formula A and a fibrate to be provided in a single pharmaceutical composition with a pharmaceutical acceptable carrier (such as in a single capsule form) designed to increase compliance and hence effectiveness. In one embodiment, the present disclosure further provides a pharmaceutical composition comprising an effective amount of the compound of formula A and an effective amount of at least one fibrate together with one or more pharmaceutically acceptable carriers, diluents, adjuvants or excipients.

In the methods of the present invention the active substances may be administered in single daily doses, or in two, three, four or more identical or different divided doses per day, and they may be administered simultaneously or at different times during the day.

In one embodiment, a compound of formula A and a fibrate(s) are administered concurrently. For example, a compound of formula A and a fibrate(s) are administered together in a single pharmaceutical composition with a pharmaceutical acceptable carrier. In another embodiment, a compound of formula A and a fibrate(s) are administered sequentially. For example, a compound of formula A is administered prior or subsequent to a fibrate(s).

In one embodiment, the active substances of the present combination are administered simultaneously, for example, as two separate dosage forms or in a single combined dosage form.

In one embodiment, a compound of formula A is administered at a first dose for a first time period, followed by administration of a compound of formula A at a second dose for a second time period. In one embodiment, a compound of formula A or a pharmaceutically acceptable salt or amino acid conjugate thereof is administered in a daily total amount from 0.1-1500 mg, 0.2-1200 mg, 0.3-1000 mg, 0.4-800 mg, 0.5-600 mg, 0.6-500 mg, 0.7-400 mg, 0.8-300 mg, 1-200 mg, 1-100 mg, 1-50 mg, 1-30 mg, 4-26 mg, or 5-25 mg for a first time period, followed by administration of a compound of formula A in a daily total amount from 0.1-1500 mg, 0.2-1200 mg, 0.3-1000 mg, 0.4-800 mg, 0.5-600 mg, 0.6-500 mg, 0.7-400 mg, 0.8-300 mg, 1-200 mg, 1-100 mg, 1-50 mg, 1-30 mg, 4-26 mg, or 5-25 mg. In one embodiment, the total amount is orally administered once a day. In one embodiment, the first dose is different from the second dose. In a further embodiment, the first dose is lower than the second dose. In another embodiment, the first dose is higher than the second dose. In one embodiment, the first dose is about 5 mg (e.g., from 4.8 mg to 5.2 mg), and the second dose is about 10 mg (e.g., from 9.8 mg to 10.2 mg). In one embodiment, the first time period is about 6 months. In one embodiment, the second time period is about 6 months.

In one embodiment, the pharmaceutical composition is administered orally, parenterally, or topically. In another embodiment, the pharmaceutical composition is administered orally.

A composition in accordance with the present invention typically contains sufficient compound of formula A or a pharmaceutically acceptable salt or amino acid conjugate thereof and a fibrate(s) to permit the desired daily dose of each to be administered to a subject in need thereof in a single unit dosage form, such as a tablet or capsule, or in two or more unit dosage forms to be administered simultaneously or at intervals during a day.

In one aspect, the two-way combination of a compound of formula A (e.g., OCA) and fibrate(s) (e.g., bezafibrate) is administered for the treatment or prevention of a disease or condition, in place of UDCA to a subject who has an inadequate therapeutic response to UDCA (used alone or in combination with another active).

In one aspect, the compound of formula A and the fibrate(s) are administered at dosages substantially the same as the dosages at which they are administered in the respective monotherapies. In one aspect, the compound of formula A is administered at a dosage which is less than (e.g., less than 90%, less than 80%, less than 70%, less than 60%, less than 50%, less than 40%, less than 30%, less than 20%, or less than 10%) its monotherapy dosage. In one aspect, the fibrate(s) is administered at a dosage which is less than (e.g., less than 90%, less than 80%, less than 70%, less than 60%, less than 50%, less than 40%, less than 30%, less than 20%, or less than 10%) its monotherapy dosage. In one aspect, both the compound of formula A and fibrate(s) are administered at a dosage which is less than (e.g., less than 90%, less than 80%, less than 70%, less than 60%, less than 50%, less than 40%, less than 30%, less than 20%, or less than 10%) their respective monotherapy dosages.

A pharmaceutical composition of the present invention may be in any convenient form for oral administration, such as a tablet, capsule, powder, lozenge, pill, troche, elixir, lyophilized powder, solution, granule, suspension, emulsion, syrup or tincture. Slow-release, modified release, or delayed-release forms may also be prepared, for example in the form of coated particles, multi-layer tablets, capsules within capsules, tablets within capsules, or microgranules.

Solid forms for oral administration may contain pharmaceutically acceptable binders, sweeteners, disintegrating agents, diluents, flavoring agents, coating agents, preservatives, lubricants and/or time delay agents. Suitable binders include gum acacia, gelatin, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavoring agents include peppermint oil, oil of wintergreen, cherry, orange or raspberry flavoring. Suitable coating agents include polymers or copolymers or acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulfite. Suitable lubricants include magnesium stearate, stearic acid, sodium oleate, sodium chloride or talc. Suitable time delay agents include glyceryl monostearate or glyceryl di stearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof

Suspensions for oral administration may further include dispersing agents and/or suspending agents. Suitable suspending agents include sodium carboxymethylcellulose, methylcellulose, hydroxypropyl methylcellulose, polyvinylpyrrolidone, sodium alginate or cetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

Emulsions for oral administration may further include one or more emulsifying agents. Suitable emulsifying agents include dispersing agents as exemplified above or natural gums such as gum acacia or gum tragacanth.

Pharmaceutical compositions of the present invention may be prepared by blending, grinding, homogenizing, suspending, dissolving, emulsifying, dispersing and/or mixing an FXR agonist (e.g., a compound of formula A or OCA or its pharmaceutically acceptable salt or amino acid conjugate thereof) and at least one fibrate together with the selected excipient(s), carrier(s), adjuvant(s) and/or diluent(s).

In some embodiments, the fibrate(s) is provided either in an immediate release tablet or in a sustained release tablet. In one of the embodiments, the fibrate(s) is provided in a sustained release tablet. In one of the embodiments, it is preferable for prolonged action that the tablet is in a sustained release format.

In another embodiment, the pharmaceutical composition of the present invention comprises a capsule containing a fibrate(s) within a capsule containing a compound of formula A or a pharmaceutically acceptable salt or amino acid conjugate thereof. In this form the fibrate(s) can be presented in an immediate release form. Another mode of administration is to provide a composition containing the fibrate(s) in a sustained release form.

In one embodiment, the pharmaceutical compositions of the invention is a dosage form which comprises a compound of formula A or a pharmaceutically acceptable salt or amino acid conjugate thereof in a daily total amount of from 0.1-1500 mg, 0.2-1200 mg, 0.3-1000 mg, 0.4-800 mg, 0.5-600 mg, 0.6-500 mg, 0.7-400 mg, 0.8-300 mg, 1-200 mg, 1-100 mg, 1-50 mg, 1-30 mg, 4-26 mg, or 5-25 mg. In one embodiment, the total amount is orally administered once a day.

In one embodiment, the pharmaceutical composition of the invention is a dosage form which comprises a fibrate in a daily total amount of 10-1000 mg, 20-800 mg, 50-500 mg, 80-400 mg, or 100-300 mg, more typically about 200 mg. In one embodiment, the total amount is orally administered once a day.

In embodiments, the composition of the invention is a dosage form which comprises a fibrate (e.g., bezafibrate) in an amount of 10-1000 mg, 20-800 mg, 50-500 mg, 80-400 mg, or 100-300 mg, more typically about 200 mg, contained within a capsule which contains the compound of formula A (e.g., OCA or compound 1) in an amount of from 0.1-1500 mg, 0.2-1200 mg, 0.3-1000 mg, 0.4-800 mg, 0.5-600 mg, 0.6-500 mg, 0.7-400 mg, 0.8-300 mg, 1-200 mg, 1-100 mg, 1-50 mg, 1-30 mg, 4-26 mg, or 5-25 mg. In some embodiments, bezafibrate is in an amount of about 200 mg, about 150 mg, about 125 mg, about 100 mg, about 75 mg, about 50 mg, about 25 mg, about 20 mg, about 15 mg, about 10 mg, or about 5 mg.

In some embodiments, the composition of the invention is a dosage form which comprises a sustained release form of bezafibrate, in an amount of 10-1000 mg, 20-800 mg, 50-500 mg, 80-400 mg, or 100-300 mg, more typically about 200 mg, contained within a capsule which contains the compound of formula A (e.g., OCA) in an amount of from 0.1-1500 mg, 0.2-1200 mg, 0.3-1000 mg, 0.4-800 mg, 0.5-600 mg, 0.6-500 mg, 0.7-400 mg, 0.8-300 mg, 1-200 mg, 1-100 mg, 1-50 mg, 1-30 mg, 4-26 mg, or 5-25 mg.

In one embodiment, the pharmaceutical composition of the present invention (the pharmaceutical combination of compound of formula A (e.g., OCA) and the fibrate (e.g., BZF)) can be used lifelong by the patient, prolonging survival and delaying liver transplantation. The reduction of hyperlipidemia and liver enzymes ensures reduction in the development of associated vascular disease. Because of the simplified dosing, the combined therapy of the present invention can be used in adjusting (increasing or decreasing) doses, depending on a patient's weight and clinical response. In one aspect, the combined therapy provides reduced side effect profile.

A composition of the present invention that comprises a compound of formula A or a pharmaceutically acceptable salt or amino acid conjugate thereof and a fibrate can be provided as a single capsule containing the two active substances within it.

The compounds of formula A disclosed herein can be prepared by conventional methods (e.g., as described in U.S. Publication No. 2009/0062526; U.S. Pat. No. 7,138,390; WO 2006/122977; WO 2013/192097; U.S. Pat. No. 7,932,244; WO 2014/066819; WO 2014/184271; and WO 2017/062763).

### DEFINITIONS

For convenience, certain terms used in the specification, examples and appended claims are collected here.

As used herein the term “fibrate” means any of fibric acid derivatives and pharmaceutically active derivatives of 2-phenoxy-2-methylpropanoic acid useful in the methods described herein. Examples of fibrates include, but are not limited to, fenofibrate, bezafibrate, beclobrate, binifibrate, ciprofibrate, clinofibrate, clofibrate, clofibric acid, etofibrate, gemfibrozil, nicofibrate, pirifibrate, ronifibrate, simfibrate, theofibrate, tocofibrate, plafibride, etc. Examples of fibrates are also described in U.S. Pat. Nos. 3,781,328, 3,948,973, 3,869,477, 3,716,583, 3,262,580, 3,723,446, 4,058,552, 3,674,836, 3,369,025, 3,984,413, 3,971,798, 6,384,062, 7,119,198 and 7,259,186; U.S. Pub. No. 20090131395; WO2008/039829; Belgian patent no. 884722; United Kingdom patent no. 860303; and European patent application publication no. EP0607536, the entire disclosures of each of which are hereby incorporated herein by reference.

Bezafibrate (BZF), a pan-peroxisome proliferator-activated receptor (PPAR) [α, δ, γ] agonist, was originally developed for treatment of hyperlipidemia and used for the prevention of cardiovascular disease. BZF also decreases serum hepatobiliary enzyme activity in individuals with and without cardiovascular disease and thus has been identified as a potential anticholestatic agent for the treatment of PBC with an inadequate response to UDCA.

OCA is a selective FXR agonist that has been shown to effect significant reductions in ALP in patients with PBC who demonstrated no or partial response to UDCA. As such, OCA has been conditionally approved for patients with PBC in combination with UDCA for those with an inadequate response to UDCA or who are intolerant to UDCA.

Without being bound to any theory, this application relates to concomitant use of OCA and BZF which results in improved efficacy and tolerability compared to the previous PBC therapies and treatment with OCA alone.

As used herein, the term “FXR agonist” refers to any compound which activates FXR. In one aspect, an FXR agonist achieves at least 50% activation of FXR relative to CDCA, the appropriate positive control in the assay methods described in WO 2000/037077. In another aspect, an FXR agonist achieves 100% activation of FXR in the scintillation proximity assay or the HTRF assay as described in WO 2000/037077. Examples of FXR agonists include but are not limited to those described in U.S. 7,138,390; 7,932,244; 20120283234; 20120232116; 20120053163; 20110105475; 20100210660; 20100184809; 20100172870; 20100152166; 20100069367; 20100063018; 20100022498; 20090270460; 20090215748; 20090163474; 20090093524; 20080300235; 20080299118; 20080182832; 20080039435; 20070142340; 20060069070; 20050080064; 20040176426; 20030130296; 20030109467; 20030003520; 20020132223; and 20020120137.

As used herein, the term “obeticholic acid” or “OCA” refers to a compound having the chemical structure:

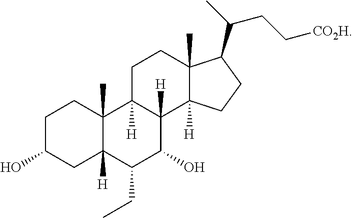

Obeticholic acid (OCA), a farnesoid X receptor (FXR) agonist and modified bile acid derived from the primary human bile acid chenodeoxycholic acid (CDCA), was developed for the treatment of PBC and to provide patients who have an inadequate response to or poor tolerance of UDCA, a novel treatment option that was safe and effective (Pellicciari 2002).

Obeticholic acid is also referred to as 3α, 7α-dihydroxy-6α-ethyl-5β-cholan-24-oic acid, 6α-ethyl-chenodeoxycholic acid, 6-ethyl-CDCA, 6ECDCA, cholan-24-oic acid, 6-ethyl-3,7-dihydroxy-(3α, 5β, 6α, 7α), and can be prepared by the methods described in U.S. Publication No. 2009/0062526 A1, U.S. Pat. No. 7,138,390, and WO2006/122977. The CAS registry number for obeticholic acid is 459789-99-2.

The term “the compound” means a compound of formula A or Compound 1, 2, or 3 (including 3a and 3b), or a pharmaceutically acceptable salt or amino acid conjugate thereof. Whenever the term is used in the context of the present invention it is to be understood that the reference is being made to a free form, an isotopically-labeled compound, a crystalline compound, a non-crystalline compound or a corresponding pharmaceutically acceptable salt or amino acid conjugates thereof, provided that such is possible and/or appropriate under the circumstances.

As used herein, the term “amino acid conjugates” refers to conjugates of a compound of the present invention (e.g., a compound of Formula A) with any suitable amino acid. For example, such a suitable amino acid conjugate of a compound of Formula A has the added advantage of enhanced integrity in bile or intestinal fluids. Suitable amino acids include but are not limited to glycine, taurine and sarcosine. Thus, the present invention encompasses the glycine, taurine and sarcosine conjugates of a compound of formula A (e.g., Compound 1).

“Treating” includes any effect, e.g., lessening, reducing, modulating, or eliminating, that results in the improvement of the condition, disease, disorder, etc. “Treating” or “treatment” of a disease state includes inhibiting the existing disease state, i.e., arresting the development of the disease state or its clinical symptoms, or relieving the disease state, i.e., causing temporary or permanent regression of the disease state or its clinical symptoms.

“Preventing” a disease state includes causing the clinical symptoms of the disease state not to develop in a subject that may be exposed to or predisposed to the disease state but does not yet experience or display symptoms of the disease state.

The term “inhibiting” or “inhibition” as used herein refers to any detectable positive effect on the progression of a disease or condition. Such a positive effect may include the delay in progression of at least one symptom or sign of the disease or condition, alleviation or reversal of the symptom(s) or sign(s) and slowing of the further worsening of the symptom(s) or sign(s).

“Disease state” means any disease, disorder, condition, symptom, or indication.

The term “effective amount” or “therapeutically effective amount” as used herein refers to an amount of an FXR-activating ligand (e.g., a compound of formula A) or a fibrate that produces an acute or chronic therapeutic effect upon appropriate dose administration, alone or in combination. In one embodiment, an effective amount or therapeutically effective amount of an FXR-activating ligand produces an acute or chronic therapeutic effect upon appropriate dose administration in combination with at least one fibrate. The effect includes the prevention, correction, inhibition, or reversal of the symptoms, signs and underlying pathology of a disease/condition (e.g., fibrosis of the liver, kidney, or intestine) and related complications to any detectable extent. An “effective amount” or “therapeutically effective amount” varies depending on the FXR agonist, the fibrate, the disease and its severity, and the age, weight, etc., of the subject to be treated.

A therapeutically effective amount of a compound of formula A can be formulated together with one or more fibrates, and optionally one or more pharmaceutically acceptable carriers for administration to a human or a non-human animal. Accordingly, the pharmaceutical composition of the invention can be administered, for example, via oral, parenteral, or topical routes, to provide an effective amount of the compound of formula A and the fibrate(s). In alternative embodiments, the compositions of the invention can be used to coat or impregnate a medical device, e.g., a stent.

“Pharmacological effect” as used herein encompasses effects produced in the subject that achieve the intended purpose of a therapy. In one embodiment, a pharmacological effect means that primary indications of the subject being treated are prevented, alleviated, or reduced. For example, a pharmacological effect would be one that results in the prevention, alleviation or reduction of primary indications in a treated subject. In another embodiment, a pharmacological effect means that disorders or symptoms of the primary indications of the subject being treated are prevented, alleviated, or reduced. For example, a pharmacological effect would be one that results in the prevention, alleviation or reduction of the disorders or symptoms in a treated subject.

It is to be understood that the isomers arising from asymmetric carbon atoms (e.g., all enantiomers and diastereomers) are included within the scope of the invention, unless indicated otherwise. Such isomers can be obtained in substantially pure form by classical separation techniques and by stereochemically controlled synthesis.

A “pharmaceutical composition” is a formulation containing therapeutic agents such as a compound of formula A and a fibrate, in a form suitable for administration to a subject. In one embodiment, the pharmaceutical composition is in bulk or in unit dosage form. It can be advantageous to formulate compositions in dosage unit form for ease of administration and uniformity of dosage. Dosage unit form as used herein refers to physically discrete units suited as unitary dosages for the subject to be treated; each unit containing a predetermined quantity of active reagent calculated to produce the desired therapeutic effect in association with the required pharmaceutical carrier. The specification for the dosage unit forms of the invention are dictated by and directly dependent on the unique characteristics of the active agents and the particular therapeutic effect to be achieved, and the limitations in the art of compounding such an active agent for the treatment of individuals.

The term “unit dosage form” refers to physically discrete units suitable as unitary dosages for humans and other mammals, each unit containing a predetermined quantity of active material calculated to produce the desired therapeutic effect, in association with a suitable pharmaceutical excipient as described herein.

The unit dosage form is any of a variety of forms, including, for example, a capsule, an IV bag, a tablet, a single pump on an aerosol inhaler, or a vial. The quantity of the compound of formula A or a pharmaceutically acceptable salt or amino acid conjugate thereof in a unit dose of composition is an effective amount and is varied according to the particular treatment involved and/or the fibrate(s) used for the treatment. One skilled in the art will appreciate that it is sometimes necessary to make routine variations to the dosage depending on the age and condition of the patient. The dosage also depends on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for topical or transdermal administration of a compound of this invention include powders, sprays, ointments, pastes, creams, lotions, gels, solutions, patches and inhalants. In one embodiment, a compound of formula A and/or fibrate is mixed under sterile conditions with a pharmaceutically acceptable carrier, and with any preservatives, buffers, or propellants that are required.

The term “flash dose” refers to formulations that are rapidly dispersing dosage forms.

The term “immediate release” is defined as a release of a therapeutic agent (such as a compound of formula A or a fibrate) from a dosage form in a relatively brief period of time, generally up to about 60 minutes. The term “modified release” is defined to include delayed release, extended release, and pulsed release. The term “pulsed release” is defined as a series of releases of drug from a dosage form. The term “sustained release” or “extended release” is defined as continuous release of a therapeutic agent from a dosage form over a prolonged period.

A “subject” includes mammals, e.g., humans, companion animals (e.g., dogs, cats, birds, and the like), farm animals (e.g., cows, sheep, pigs, horses, fowl, and the like), and laboratory animals (e.g., rats, mice, guinea pigs, birds, and the like). In one embodiment, the subject is a human. In one aspect, the subject is female. In one aspect, the subject is male.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable carrier or excipient” means a carrier or excipient that is useful in preparing a pharmaceutical composition that is generally safe, non-toxic and neither biologically nor otherwise undesirable, and includes any excipient that is acceptable for veterinary use and/or human pharmaceutical use. A “pharmaceutically acceptable excipient” as used herein includes both one and more than one such excipient.

A compound of formula A may be administered in the form of a pharmaceutical formulation comprising a pharmaceutically acceptable excipient. This formulation can be administered by a variety of routes including oral, buccal, rectal, intranasal, transdermal, subcutaneous, intravenous, intramuscular, and intranasal.

The compound of formula A may be administered over a wide dosage range. For example, dosages per day normally fall within the range of about 0.0001 to about 30 mg/kg of body weight. In the treatment of adult humans, the range of about 0.1 to about 15 mg/kg/day, in single or divided dose, may be used. In one embodiment, the formulation comprises about 0.1 mg to about 1500 mg of a compound of formula A. In another embodiment, the formulation comprises about 1 mg to about 100 mg of a compound of formula A. In another embodiment, the formulation comprises about 1 mg to about 50 mg of a compound of formula A. In another embodiment, the formulation comprises about 1 mg to about 30 mg of a compound of formula A. In another embodiment, the formulation comprises about 4 mg to about 26 mg of a compound of formula A. In another embodiment, the formulation comprises about 5 mg to about 25 mg of a compound of formula A. However, the amount of the compound of formula A (e.g., OCA) actually administered can be determined by a physician, in light of the relevant circumstances, including the condition to be treated, the chosen route of administration, the form of the compound of formula A administered, the fibrate administered, the age, weight, and response of the individual patient, and the severity of the patient's symptoms. Therefore, the invention is not limited to the above-mentioned dosage ranges. In some instances, dosage levels below the lower limit of the aforesaid range may be more than adequate, while in other cases still larger doses may be employed without causing any harmful side effect, provided that such larger doses are first divided into several smaller doses for administration throughout the day.

“Fibrosis” refers to a condition involving the development of excessive fibrous connective tissue, e.g., scar tissue, in a tissue or organ. Such generation of scar tissue may occur in response to infection, inflammation, or injury of the organ due to a disease, trauma, chemical toxicity, and so on. Fibrosis may develop in a variety of different tissues and organs, including the liver, kidney, intestine, lung, heart, etc.

As used herein, a “cholestatic condition” refers to any disease or condition in which bile excretion from the liver is impaired or blocked, which can occur either in the liver or in the bile ducts. Intrahepatic cholestasis and extrahepatic cholestasis are the two types of cholestatic conditions. Intrahepatic cholestasis (which occurs inside the liver) is most commonly seen in primary biliary cirrhosis, primary sclerosing cholangitis, sepsis (generalized infection), acute alcoholic hepatitis, drug toxicity, total parenteral nutrition (being fed intravenously), malignancy, cystic fibrosis, biliary atresia, and pregnancy. Extrahepatic cholestasis (which occurs outside the liver) can be caused by bile duct tumors, strictures, cysts, diverticula, stone formation in the common bile duct, pancreatitis, pancreatic tumor or pseudocyst, and compression due to a mass or tumor in a nearby organ.

Clinical symptoms and signs of a cholestatic condition include itching (pruritus), fatigue, jaundiced skin or eyes, inability to digest certain foods, nausea, vomiting, pale stools, dark urine, and right upper quadrant abdominal pain. A patient with a cholestatic condition can be diagnosed and followed clinically based on a set of standard clinical laboratory tests, including measurement of levels of alkaline phosphatase, y-glutamyl transpeptidase (GGT), 5′ nucleotidase, bilirubin, bile acids, and cholesterol in a patient's blood serum. Generally, a patient is diagnosed as having a cholestatic condition if serum levels of all three of the diagnostic markers: alkaline phosphatase, GGT, and 5′ nucleotidase, are considered abnormally elevated. The normal serum level of these markers may vary to some degree from laboratory to laboratory and from procedure to procedure, depending on the testing protocol. Thus, a physician is able to determine, based on the specific laboratory and test procedure, what an abnormally elevated blood level is for each of the markers. For example, a patient suffering from a cholestatic condition generally has greater than about 125 IU/L alkaline phosphatase, greater than about 65 IU/L GGT, and greater than about 17 NIL 5′ nucleotidase in the blood. Because of the variability in the level of serum markers, a cholestatic condition may be diagnosed on the basis of abnormal levels of these three markers in addition to at least one of the symptoms mentioned above, such as itching (pruritus).

Pruritus is an adverse event (AE) and must be graded for severity (i.e., intensity). Because pruritus is a subjective symptom and its occurrence and magnitude are not readily measured by objective tools, clinical judgment is applied to determine its severity and management in each subject. In order to assess the potential improvement in pruritus with treatment, baseline pruritus presence (yes/no) and severity is determined. Severity of Pruritus: 1=Mild (Mild or localized; topical intervention indicated); 2=Moderate (Intense or widespread; intermittent; skin changes from scratching (e.g., edema, papulation, excoriations, lichenification, oozing/crusts); oral intervention indicated; limiting instrumental activities of daily living); 3=Severe (Intense or widespread; constant; limiting self-care activities of daily living or sleep; oral corticosteroid or immunosuppressive therapy indicated). The present application also relates to a method of reducing adverse events, such as pruritus, comprising administering the disclosed combination. The present application also relates to a method of mitigating adverse events elicited or caused by OCA monotherapy, such as pruritus, comprising administering the disclosed combination of the compound of formula A (e.g., OCA) and a fibrate (e.g., BZF).

The term “primary biliary cholangitis”, previously called “primary biliary cirrhosis”, often abbreviated PBC, is an autoimmune disease of the liver marked by the slow progressive destruction of the small bile ducts of the liver, with the intralobular ducts (Canals of Hering) affected early in the disease. When these ducts are damaged, bile builds up in the liver (cholestasis) and over time damages the tissue. This can lead to scarring, fibrosis and cirrhosis. Primary biliary cirrhosis is characterized by interlobular bile duct destruction. Histopathologic findings of primary biliary cirrhosis include: inflammation of the bile ducts, characterized by intraepithelial lymphocytes, and periductal epithelioid granulomata. There are 4 stages of PBC.
Stage 1—Portal Stage: Normal sized triads; portal inflammation, subtle bile duct damage. Granulomas are often detected in this stage.
Stage 2—Periportal Stage: Enlarged triads; periportal fibrosis and/or inflammation. Typically, this stage is characterized by the finding of a proliferation of small bile ducts.
Stage 3—Septal Stage: Active and/or passive fibrous septa.
Stage 4—Biliary Cirrhosis: Nodules present; garland

The term “primary sclerosing cholangitis” (PSC) is a disease of the bile ducts that causes inflammation and subsequent obstruction of bile ducts both at an intrahepatic (inside the liver) and extrahepatic (outside the liver) level. The inflammation impedes the flow of bile to the gut, which can ultimately lead to cirrhosis of the liver, liver failure and liver cancer.

The term “organ” refers to a differentiated structure (as in a heart, lung, kidney, liver, etc.) consisting of cells and tissues and performing some specific function in an organism. This term also encompasses bodily parts performing a function or cooperating in an activity (e.g., an eye and related structures that make up the visual organs). The term “organ” further encompasses any partial structure of differentiated cells and tissues that is potentially capable of developing into a complete structure (e.g., a lobe or a section of a liver).

All publications and patent documents cited herein are hereby incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the description and examples provided herein are for purposes of illustration and not limitation of the claims that follow.

In the specification, the singular forms also include the plural, unless the context clearly dictates otherwise. Unless defined otherwise, all technical and scientific terms used herein have the same meaning as commonly understood by one of ordinary skill in the art to which this invention belongs. In the case of conflict, the present specification controls. All percentages and ratios used herein, unless otherwise indicated, are by weight.

### EXAMPLES

### Example 1: Clinical Trial to Determine The Effects of OCA in Combination with BZF on PBC

### Study Design

A phase 2, double-blind (DB), randomized, parallel-group study evaluating the efficacy, safety, and tolerability of obeticholic acid (OCA), administered alone or in combination with bezafibrate (BZF) is being conducted in subjects with primary biliary cholangitis (PBC) who have an inadequate response or who are unable to tolerate ursodeoxycholic acid (UDCA). This study evaluates the efficacy, safety, and tolerability of OCA alone or in combination with 2 different BZF doses in approximately 54 subjects with PBC over at least 12 weeks.

The primary outcome measure is to assess the effects of the combination of OCA and BZF on ALP in comparison to OCA alone in subjects with PBC who have an inadequate response or who are unable to tolerate UDCA.

The secondary outcomes are to assess the effects of the combination of OCA and BZF in comparison to OCA alone in subjects with PBC who have an inadequate response or who are unable to tolerate UDCA on the following: (1) safety and tolerability, (2) response and normalization rates of biochemical disease markers, (3) disease-specific symptoms as assessed by health-related quality of life questionnaires, and (4) biomarkers of bile acid synthesis and homeostasis, including 7a hydroxy 4 cholesten-3-one (C4) and bile acids.

The additional objectives are to assess the effects of the combination of OCA and BZF in comparison to OCA alone in subjects with PBC who have an inadequate response or who are unable to tolerate UDCA on the following: (1) noninvasive assessments of liver fibrosis (transient elastography [TE] and markers of collagen formation and degradation [type III pro-collagen (Pro-C3), type V pro-collagen (Pro-05), type III collagen (C3M), and type IV collagen (C4M)]), (2) estimated long-term prognosis (GLOBE and UK-PBC scores), (3) safety (model of end-stage liver disease [MELD] score, physical examinations, electrocardiograms [ECGs], and vital signs), (4) the PK of BZF and OCA and its conjugates, glyco-OCA and tauro-OCA, and (5) PK/pharmacodynamics (PD) and PK/safety relationships.

### Inclusion and Exclusion Criteria

PRINCIPAL INCLUSION CRITERIA include but are not limited to:
(1) a definite or probable diagnosis of PBC (consistent with the European Association for the Study of the Liver [EASL] Practice Guidelines and the American Association for the Study of Liver Diseases; [Lindor 2009a, EASL 2017]), as demonstrated by the presence of at least 2 of the following 3 diagnostic factors: (a) history of elevated ALP levels for at least 6 months, (b) positive antimitochondrial antibody (AMA) titer, or if AMA negative or low titer (≤1:80), (c) PBC specific antibodies (anti-GP210 and/ or anti-SP100), (d) antibodies against the major M2 components (pyruvate dehydrogenase-E2, 2-oxo-glutaric acid dehydrogenase complex), and (e) Liver biopsy results consistent with PBC (collected at any time before Screening),
(2) at least one of the following qualifying biochemistry values: (a) ALP>1.5×ULN (including a maximum of 25% of subjects with ALP>1.5 ULN and ≤1.67 ULN) enrolled in the study and/or (b) total bilirubin>ULN but <2×ULN,
(3) age≥18 years,
(4) taking UDCA for at least 12 months (stable dose for ≥3 months) before Day 1 or unable to tolerate or unresponsive to UDCA (no UDCA for ≥3 months) before Day 1,
(5) must provide written informed consent and agree to comply with the study protocol.

PRINCIPAL EXCLUSION CRITERIA include but are not limited to:
(1) history or presence of other concomitant liver diseases, including the following:
   Hepatitis C virus (HCV) infection and ribonucleic acid positive
   Active hepatitis B virus (HBV) infection; however, subjects who have seroconverted (hepatitis B surface antigen and hepatitis B antigen negative) may be included in this study after consultation with the Medical Monitor
   Primary sclerosing cholangitis
   Alcoholic liver disease
   Definite autoimmune liver disease or overlap hepatitis
   NASH
   Gilbert's Syndrome (due to interpretability of bilirubin levels)
(2) presence of clinical complications of PBC or clinically significant (CS) hepatic decompensation at Screening Visit 1 and 2, including the following:
   History of liver transplantation
   Current placement on a liver transplant list, although subjects who are placed on a transplant list despite a relatively early disease stage (e.g., per regional guidelines) may be eligible as long as they do not meet any of the other exclusion criteria
   Current CP Grade B or C (i.e., CP score >6)
   Portal hypertension with complications, including known gastric or large esophageal varices, poorly controlled or diuretic-resistant ascites, history of variceal bleeds or related therapeutic or prophylactic interventions (e.g., beta blockers, insertion of variceal bands or transjugular intrahepatic portosystemic shunts [TIPS]), or hepatic encephalopathy
   Cholangitis with complications, including history or presence of spontaneous bacterial peritonitis, hepatocellular carcinoma, or bilirubin >2×ULN
(3) medical conditions that may cause nonhepatic increases in ALP (e.g., Paget's disease) or which may diminish life expectancy to <2 years, including known cancers (except carcinomas in situ or other stable, relatively benign conditions),
(4) presence of any other disease or condition that interferes with the absorption, distribution, metabolism, or excretion of drugs including bile salt metabolism in the intestine (e.g., inflammatory bowel disease or gastric bypass procedure [gastric lap band is acceptable])
(5) current or history of gallbladder disease with or without cholelithiasis and symptoms,
(6) history of drug-induced myopathy,
(7) severe renal failure (serum creatinine>1.5 mg/100 ml (>135 μmol/L); creatinine clearance<60 ml/min) or undergoing dialysis,
(8) platelet count<100 000/ml at Screening Visit 1 and 2,
(9) known history of human immunodeficiency virus (HIV) infection,
(10) history or presence of clinically concerning cardiac arrhythmias likely to affect survival during the study, or Screening (pretreatment) QT or QTc interval of >500 milliseconds,
(11) severe pruritus or required systemic treatment for pruritus (e.g., with bile acid sequestrants or rifampicin) within 2 months of Day 1,
(12) history of known or suspected CS hypersensitivity to OCA, BZF, or other fibrates or any of their components,
(13) known photoallergic or phototoxic reactions to fibrates,
(14) if female, known pregnancy, or has a positive urine pregnancy test (confirmed by a positive serum pregnancy test), or lactating,
(15) other CS medical conditions that are not well controlled or for which medication needs are anticipated to change during the study (e.g., type 2 diabetes mellitus, hypothyroidism, nephritic syndrome, dysproteinemia, obstructive liver disease),
(16) treatment with the following medications 30 days before Day 1 or plans to use these medications during the study: azathioprine, colchicine, cyclosporine, methotrexate, mycophenolate mofetil, pentoxifylline, statins, budesonide and other systemic corticosteroids, monoamine oxidase inhibitors (MAOIs), and potentially hepatotoxic drugs (including α-methyl-dopa, sodium valproic acid, isoniazide, and nitrofurantoin),
(17) treatment with the following medications 12 months before Day 1 or plans to use these medications during the study: antibodies or immunotherapy directed against interleukins or other cytokines or chemokines,
(18) participation in another investigational product, biologic, or medical device study within 30 days before Screening,
(19) known photoallergic or phototoxic reactions to fibrates,
(20) previously treated with commercially available OCA or participated in a previous study involving OCA within 6 months before Screening or plans to use commercially available OCA during the study,
(21) unable to tolerate BZF or other fibrates, was treated with commercially available fibrates or participated in a previous study involving fibrates within 3 months before Screening, or plans to use commercially available fibrates during the study,
(22) history of or ongoing alcohol or drug abuse within 1 year before Day 1
(23) history of noncompliance with medical regimens, or is considered to be potentially unreliable,
(24) blood or plasma donation within 30 days before Day 1,
(25) mental instability or incompetence, such that the validity of informed consent or ability to be compliant with the study is uncertain, and
(26) a CK value at screening >5×ULN or any abnormal laboratory value that is considered CS.

### Outcomes/End Points

Primary End Point (can be repeated as necessary): absolute change in ALP from baseline to Week 12 in the DB Treatment Period. This end point is evaluated at week 12. Secondary End Point (can be repeated as necessary): the effects of the combination of OCA and BZF in comparison to OCA alone in subjects with PBC who have an inadequate response or who are unable to tolerate UDCA on the following: (a) safety and tolerability, (b) response and normalization rates of biochemical disease markers, (c) disease-specific symptoms as assessed by health-related quality of life questionnaires, and (d) biomarkers of bile acid synthesis and homeostasis. This end point is evaluated at the end of the study.

### Screening Period

Subjects are screened for a period of 2 to 8 weeks before entering the study to allow for the collection of repeat serum chemistry samples (at least 2 weeks apart) for verification of inclusion/exclusion criteria and establishing baseline.

### DB Treatment Period (at least 12 Weeks)

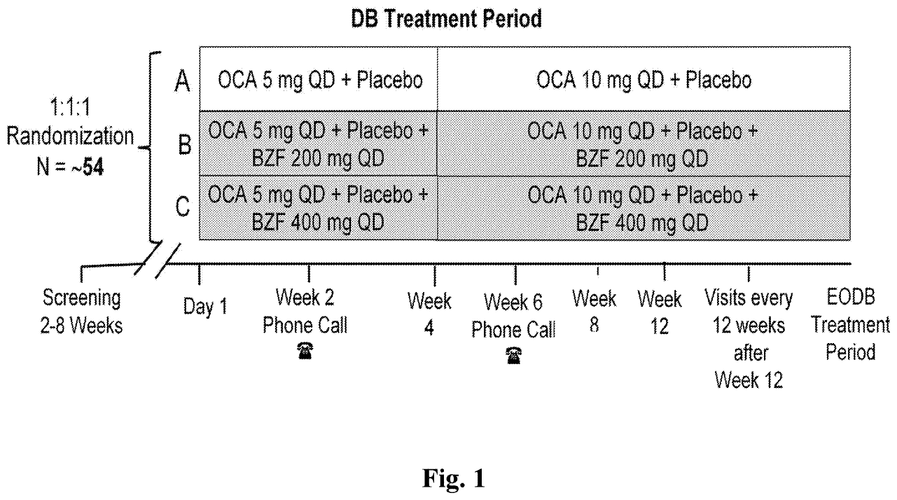

Subjects who meet the entry requirements are randomized in a 1:1:1 ratio on Day 1 to receive OCA alone and in combination with 1 of 2 BZF/placebo regimens in conjunction with standard-of-care OCA titration: Treatment A: OCA 5 mg→>10 mg QD, Treatment B: OCA 5 mg→>10 mg QD+BZF200 mg IR QD, or Treatment C: OCA 5 mg→10 mg QD+BZF 400 mg SR QD. All subjects are administered 5-mg doses of OCA QD from Day 1 to the day before the Week 4 Visit, followed by 10-mg doses of OCA QD from the Week 4 Visit through the end of the study. Dose adjustments based on ALP normalization and tolerability concerns are allowed as described. To preserve the study blind, appearance-matched placebo tablets for BZF are administered to subjects in each treatment group from Day 1 to Week 12 as shown in Diagram 1 and FIG. 1 (EODB=end of DB. Note: subjects taking UDCA at the time of enrollment remain on their stable dose of UDCA during the study, and the DB treatment continues until all subjects complete Week 12 in the DB Treatment Period).

Diagram 1

| Treatment | DB Treatment Morning Dose Regimen |
|---|---|
| Treatment A: | 1 OCA 5 mg tablet (Weeks 0 to 4) |
| OCA 5 mg→10 mg QD | 1 OCA 10 mg tablet (Week 4 to end of |
| | DB treatment) |
| | 1 placebo BZF 200 mg IR tablet |
| | 1 placebo BZF 400 mg SR tablet |
| Treatment B: | 1 OCA 5 mg tablet (Weeks 0 to 4) |
| OCA 5 mg→10 mg QD + BZF 200 mg IR | 1 OCA 10 mg tablet (Week 4 to end of |
| QD | DB treatment) |
| | 1 BZF 200 mg IR tablet |
| | 1 placebo BZF 400 mg SR tablet |
| Treatment C: | 1 OCA 5 mg tablet (Weeks 0 to 4) |
| OCA 5 mg→10 mg QD + BZF 400 mg SR | 1 OCA 10 mg tablet (Week 4 to end of |
| QD | DB treatment) |
| | 1 placebo BZF 200 mg IR tablet |
| | 1 BZF 400 mg SR tablet |

BZF = bezafibrate; DB = double-blind; IR = immediate release; OCA = obeticholic acid; QD = once daily; SR = sustained release.

Randomization is stratified by total bilirubin levels at baseline (≤0.7× or >0.7×ULN). In addition, the proportion of subjects enrolled in the study with ALP >1.5 ULN and 1.67 ULN at baseline does not exceed 25% of the overall study population.

After the Day 1 Visit, subsequent clinic visits during the DB Treatment Period occurs at approximately Weeks 4, 8, and 12 and then every 12 weeks for the assessment of efficacy, safety, tolerability, and PK. Subjects are also contacted by telephone at Weeks 2 and 6 (±5 days) to assess for occurrence of any AEs, changes to concomitant medications and/or new medications that have been initiated, and medical/surgical procedures, and to verify that the subject is dosing as directed. An evaluation of available efficacy and safety data may occur during both the DB and LTSE phases.

### Long-Term Safety Extension (LTSE) Period (up to 48 Weeks)

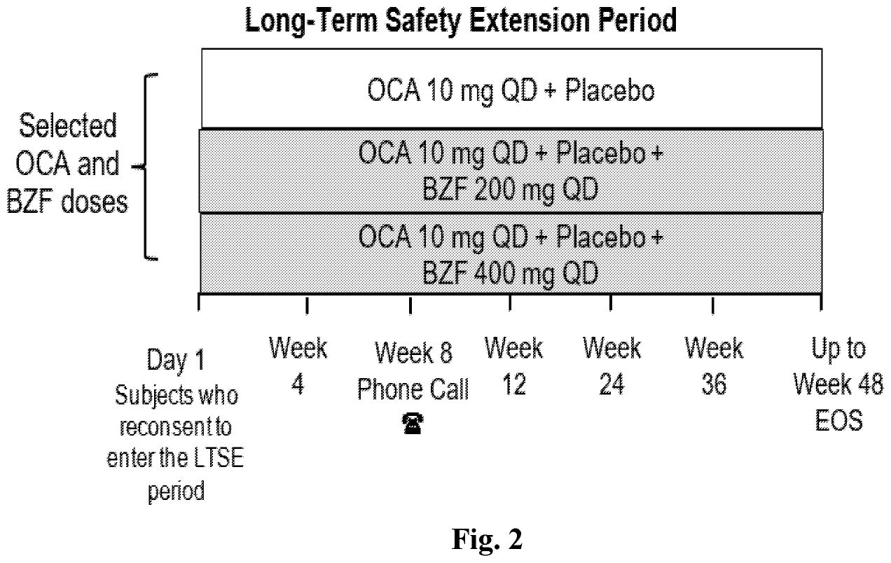

All randomized subjects continue DB treatment until the last subject complete the 12-week DB Treatment period. Subjects enter the LTSE and continue the original treatment assignment allocated during the DB phase for the remainder of the LTSE component. During the LTSE period, the dose may be optimized based on an assessment of safety and efficacy during the DB phase, in which case the protocol is amended, and subjects are transitioned to this dose after appropriate informed consent is obtained. Safety and laboratory assessments are evaluated at clinic visits once every 12 weeks and up to Week 48. The study design diagram of LTSE is shown in FIG. 2 (EOS=end of study/end of LTSE Period. Note: subjects taking UDCA at the time of re-consent remain on their stable dose of UDCA during the study).

### Study Duration

The total duration of treatment is approximately 72 weeks and is determined by the time required for all subjects to complete the DB Treatment Period, which is anticipated to be a total of 24 weeks, followed by up to 48 weeks of treatment during the LTSE period.

### Number of Subjects

Approximately 54 subjects (18 per group), including a maximum of 25% of subjects with baseline ALP>1.5×ULN and ≤1.67×ULN, are enrolled and randomized in a 1:1:1 ratio to 1 of 3 treatment arms (Treatments A:B:C). Randomization is stratified by total bilirubin levels at baseline (≤0.7× or >0.7×ULN).

### Dosing Regimen

Subjects are randomly assigned in a 1:1:1 ratio to receive the following treatments during the DB Treatment Period. All randomized subjects continue DB treatment until the last subject complete the 12-week DB Treatment period. Subjects enter the LTSE and continue the original treatment assignment allocated during the DB phase for the remainder of the LTSE phase. During the LTSE period, the dose may be optimized based on an assessment of safety and efficacy during the DB phase, in which case the protocol is amended, and subjects are transitioned to this dose after appropriate informed consent is obtained.

### Monitoring and Management of Potential Hepatic Injury and/or Disease Progression

Given the chronic and progressive nature of PBC, it is important to monitor for potential hepatic injury, disease progression, and/or hepatic decompensation. Child-Pugh and MELD scores are reviewed at each visit where labs are drawn. Child Pugh Scores are only applied in patients who have evidence of cirrhosis at screening or demonstrate evidence of cirrhosis at screening or progression to cirrhosis during the study based on known criteria. In addition, adverse events (AEs), signs and symptoms of potential hepatic injury or decompensation, and laboratory values are reviewed at regular intervals. Based on the assessments of signs and symptoms of hepatic injury and liver biochemistry, the investigational product (OCA or BZF) may be interrupted or discontinued.

### Dosage Adjustment Criteria

Double-Blind Period: With an exception of the planned dose of 5 mg OCA from Week 1 through Week 4, dosages for investigational products are maintained constant during the study. However, dose frequency may be modified for the management of pruritus or other safety findings. In the event of tolerability issues such as pruritus, the dosing frequency may be decreased. Subjects can be discontinued from the investigational product at any time for clinical safety concerns.

LTSE Period: All randomized subjects continue DB treatment until the last subject has completed the 12-week DB Treatment period. Subjects enter the LTSE and receive the original treatment that they received during the DB phase for the remainder of the LTSE phase. During the LTSE period, the dose may be optimized based on an assessment of safety and efficacy during the DB phase, in which case the protocol is amended, and subjects are transitioned to this dose after appropriate informed consent is obtained. Dose frequency may be modified for the management of pruritus or other safety findings. In the event of tolerability issues such as pruritus, the dosing frequency may be decreased. Subjects can be discontinued from the investigational product at any time for clinical safety concerns.

### Overview of Assessments

Criteria for Evaluation:

| | |
|---|---|
| Analysis Variables | Endpoint Assessments |

Primary Objectives

| | |
|---|---|
| Change in ALP | Absolute change in ALP from baseline to |
| | Week 12 in the DB Treatment Period |

Secondary Objectives

| | |
|---|---|
| Safety and tolerability | SAEs, TEAEs, clinical laboratory assessments, |
| | early discontinuations, and eGFR |
| Response and normalization rates of | ALP response rates of 10%, 20%, and 40% |
| biochemical disease markers | change and normalization rates |
| | Clinical laboratory values: |
| | ALP, GGT, ALT, AST, and total and |
| | conjugated bilirubin, APRI |
| Disease-specific symptoms assessed by | PBC-40, pruritus VAS, EQ-5D-5L (EuroQol |
| health-related quality of life questionnaires | five dimensions questionnaire), and SF-36 |
| | (Short Form Health Survey) |
| Biomarkers of bile acid synthesis and | C4 and bile acids |
| homeostasis | |

Additional Objectives

| | |
|---|---|
| Noninvasive assessments of liver fibrosis | TE and markers of collagen formation and |
| | degradation (Pro-C3, Proc-C5, C3M, and C4M) |
| Estimated long-term prognosis | GLOBE (a risk score used to predict |
| | transplantation-free survival) and UK-PBC |
| | scores |
| Safety | MELD (model of end-stage liver disease) score, |
| | physical examinations, ECGs, and vital signs |
| PK parameters | Cₘₐₓ, Tₘₐₓ, AUC, t_{1/2} of BZF and OCA and its |
| | conjugates, glyco-OCA and tauro-OCA |
| PK/PD and PK/safety relationships | PK parameters compared to PD parameters and |
| | safety and tolerability assessments (above) |

ALP = alkaline phosphatase; ALT = alanine aminotransferase; APRI = aspartate aminotransferase to platelet ratio index; AST = aspartate aminotransferase; AUC = area under the concentration-time curve; BZF = bezafibrate; C4 = 7α-hydroxy-4-cholesten-3-one; Cₘₐₓ = peak (maximum) plasma concentration; ECG = electrocardiogram; eGFR = estimated glomerular filtration rate; GGT = gamma-glutamyl transferase; MELD = model of end-stage liver disease; OCA = obeticholic acid; PBC = primary biliary cholangitis; PD = pharmacodynamics; PK = pharmacokinetic; SAE = serious adverse event; t_{1/2} = half-life; TE = transient elastography; TEAE = treatment-emergent adverse event; Tₘₐₓ = time to Cₘₐₓ; UK = United Kingdom; VAS = visual analog scale.

### Analysis Populations

Evaluable Population—all subjects who complete the DB Treatment Period and have adequate exposure to investigational products (OCA and/or BZF) without any major protocol deviations.

ITT (Intent-to-Treat) Population—all randomized subjects who receive at least 1 dose of OCA and/or BZF. Treatment assignment is based on the randomized treatment.

Safety Population—all randomized subjects who receive at least 1 dose of OCA and/or BZF. Treatment assignment is based on the treatment actually received.

Pharmacokinetic Population—all subjects who receive OCA and/or BZF and have at least 1 confirmed analyzable sample. Subjects must not have any major protocol deviations that potentially affect exposure levels.

LTSE (long-term safety extension) Population—all subjects who receive at least 1 dose of OCA and/or BZF during the LTSE period.

### Efficacy Analyses

Primary Efficacy Analysis—The Evaluable Population is the primary population used for the efficacy analyses. The primary efficacy endpoint is absolute change in ALP from baseline to Week 12 in the DB Treatment Period. Analyses of change in ALP are carried out using an analysis of covariance (ANCOVA) model at Week 12 with change from baseline as the dependent variable, treatment group and randomization stratification factor as fixed effects, and the baseline values as a covariate. Estimates of least squares (LS) means, standard errors (SEs), and 95% confidence intervals (CIs) are presented by treatment group. Estimates of the mean difference between treatment groups, the SE of the difference, and 95% CI of the difference are presented. The same analysis is carried out using percent change from baseline as the dependent variable. Comparison of treatment groups is based on their mean estimates and the associated 95% CIs; no formal hypothesis testing is planned. An optimal treatment arm may be identified/selected based on the consistency of results for a set of efficacy biochemical parameters.

Secondary and Additional Efficacy Analyses—The Evaluable Population is the primary population used for the secondary and additional efficacy analyses. Secondary and additional efficacy analyses are summarized using descriptive statistics at Baseline and at each scheduled postbaseline visit comparing the OCA and OCA+BZF treatment groups. The change from baseline and percent change from baseline is also summarized. Descriptive statistics, including change from baseline, percent change from baseline, and estimates of LS means, standard errors, and 95% CIs, are presented by treatment group. Estimates of the mean difference between treatment groups, the SE of the difference, and 2-sided 95% CI of the difference are also presented. Analyses for ALP response rates of 10%, 20%, and 40% change and normalization rates compare the OCA +BZF treatment groups to the OCA treatment group using a Cochran-Mantel-Haenszel test stratified by the randomization stratification factor. The PBC-40, pruritus VAS, EQ-5D-5L, and SF-36 are compared between the OCA +BZF treatment groups and the OCA treatment group using a Wilcoxon rank-sum test.

### Pharmacokinetic Analyses

The PK Population is the primary population used for PK, PK/PD, and PK/safety analyses. PK parameter estimates are determined for plasma BZF and unconjugated OCA (parent), glyco-OCA, tauro-OCA, and total OCA (sum of OCA, glyco-OCA, and tauro-OCA) using standard noncompartmental methods based on actual sample collection times.

### PK/PD and PK/Safety Analyses

The PK/PD relationship of total OCA and/or BZF PK exposure parameters versus C4, total endogenous bile acids, and ALP are evaluated. The PK/safety relationship of total OCA and/or BZF PK exposure parameters versus pruritus and liver biochemistry markers (e.g., ALP) are evaluated.

### Safety Analyses

The Safety Population is the primary population used for safety analyses. Treatment assignment is based on the treatment actually received. Safety data, including serious AEs (SAEs), treatment-emergent AEs (TEAEs), physical examinations, electrocardiograms (ECGs), vital signs, clinical laboratory assessments, and treatment discontinuations are compared across all treatment groups during the DB Treatment Period. The incidence of TEAEs and SAEs is tabulated by System Organ Class (SOC) and Preferred Term for each treatment group and similarly by severity and relationship to treatment. Laboratory parameters and vital signs are summarized by treatment group using descriptive statistics at Baseline and at each scheduled postbaseline visit. The change from baseline is also summarized. ECGs are summarized by treatment group using frequency at each visit. The shift from baseline is also summarized. Baseline is defined as the mean of all available evaluations before treatment.

### LTSE Analyses

Similar analyses to that described for the DB Treatment Period are conducted for the LTSE Period data using the DB baseline value (with the exception of PK, which is not performed during the LTSE Period). Analyses based on the DB baseline are performed using randomized treatment groups.

### Interim Analysis

No interim analysis with formal statistical hypothesis testing or with an intention of stopping the study early is planned. Additional evaluations of available efficacy and safety data may be conducted during the LTSE phase, at approximately the time when all subjects have completed Weeks 12, 24, 36 and 48.

### Sample Size Justification

A sample size of 18 subjects per treatment group provides at least 80% power to detect a treatment difference for change in ALP of 60 U/L, assuming that the mean absolute changes in ALP for OCA+BZF and OCA treatment groups are approximately −160 and −100 U/L, respectively, with a pooled standard deviation of 58 U/L and a 10% dropout rate, based on a 2-sided independent 2-group t-test at an alpha level of 0.05.

Example 2: Clinical trial to assess the effects of the combination of OCA and BZF in comparison to BZF alone in subjects with PBC

### Study Design

A Phase 2, double-blind (DB), randomized, parallel group study evaluating the efficacy, safety, and tolerability of obeticholic acid (OCA) administered in combination with Bezafibrate (BZF) in subjects with primary biliary cholangitis (PBC) who have an inadequate response or who are unable to tolerate ursodeoxycholic acid. This study assesses the effects of the combination of OCA and BZF in comparison to BZF alone in subjects with PBC. OCA (5 mg and 10 mg) in combination with 2 different BZF doses (400 mg and 200 mg) or BZF alone (at two doses, 200 mg and 400 mg) is administered to 72 subjects with PBC over at least 12 weeks.

The primary outcome measure is to assess the effects of the combination of OCA and BZF on ALP in comparison to BZF alone in subjects with PBC.

The secondary outcomes are to assess the effects of the combination of OCA and BZF in comparison to BZF alone in subjects with PBC on the following: (1) response and normalization rates of biochemical disease markers; (2) disease-specific symptoms as assessed by health-related quality of life questionnaires (PBC-40, pruritus visual analog scale [VAS], EQ-5D-5L, and SF-36); (3) biomarkers of bile acid synthesis and homeostasis, including 7a-hydroxy-4-cholesten-3-one (C4) and bile acids; and (4) safety and tolerability.

The additional objectives are to assess the combination of OCA and BZF in comparison to BZF alone in subjects with PBC on the following: (1) noninvasive assessments of liver fibrosis (transient elastography [TE], enhanced liver fibrosis [ELF]); and markers of collagen formation and degradation (type III pro-collagen [Pro-C3], type V pro-collagen [Pro-C5], type III collagen [C3M], and type IV collagen [C4M]); (2) noninvasive assessment of liver function (HepQuant SHUNT); (3) estimated long-term prognosis (GLOBE and UK-PBC scores); (4) MELD score; (5) pharmacokinetics (PK) of BZF (and its metabolites, which may include BZF-glucuronide and BZF-hydroxide) and OCA and its conjugates, glyco-OCA and tauro-OCA; and (6) PK/pharmacodynamics (PD) and PK/safety relationships.

### Inclusion and Exclusion Criteria

PRINCIPAL INCLUSION CRITERIA include but are not limited to:
(1) a definite or probable diagnosis of PBC (consistent with the EASL and the AASLD guidelines [Lindor 2009a, EASL 2017]), as demonstrated by the presence of at least 2 of the following 3 diagnostic factors: (a) history of elevated ALP levels for at least 6 months; (b) positive antimitochondrial antibody (AMA) titer, or if AMA negative or low titer (≤1:80), PBC-specific antibodies (anti-GP210 and/or anti-SP100) and/or antibodies against the major M2 components (pyruvate dehydrogenase-E2, 2-oxo-glutaric acid dehydrogenase complex); (c) liver biopsy results consistent with PBC (collected at any time before Screening);
(2) at least one of the following qualifying biochemistry values (the mean of both screening visits): (a) ALP >1.5×ULN (including a maximum of 25% of patients with ALP>1.5 ULN but ≤1.67 ULN) will be enrolled in the study; (b) total bilirubin>ULN but<2×ULN;
(3) age≥18 years;
(4) taking UDCA for at least 12 months (stable dose for ≥3 months) before Day 1 or unable to tolerate or unresponsive to UDCA (no UDCA for ≥3 months before Day 1);
(5) contraception: Female subjects must be postmenopausal, surgically sterile, or, if premenopausal (and not surgically sterile), be prepared to use >1 highly effective method of contraception during the study and for 30 days after the end of treatment. Highly effective methods of contraception per the Clinical Trials Facilitation and Coordination Group (CTFG) guidelines are those that alone or in combination results in a failure rate of less than 1% per year when used consistently and correctly. Highly effective methods of contraception are as follows: (a) Intrauterine device (e.g., intrauterine device (IUD) or intrauterine hormone-releasing system (IUS)); (b) Bilateral tubal occlusion; (c) Vasectomy (partner); (d) Combined (estrogen and progestogen containing) hormonal contraception (e.g., oral, intravaginal or transdermal) associated with inhibition of ovulation. If oral contraceptives are used, they must be used in combination with a male or female condom. Female subjects should be on hormonal contraception for at least 8 days prior to Day 1; (e) Progestogen-only hormonal contraception (e.g., oral, injectable or implantable) associated with inhibition of ovulation. If oral contraceptives are used, they must be used in combination with a male or female condom. Female subjects should be on hormonal contraception for at least 8 days prior to Day 1; (f) Sexual abstinence, if in line with the preferred and usual lifestyle of the subject (where abstinence is defined as refraining from heterosexual intercourse during the entire period of risk associated with study treatments); and
(6) must provide written informed consent and agree to comply with the study protocol.

PRINCIPAL EXCLUSION CRITERIA include but are not limited to:
(1) history or presence of other concomitant liver diseases including the following: (a) Hepatitis C virus (HCV) infection and ribonucleic acid positive; (b) Active hepatitis B virus (HBV) infection; however, subjects who have seroconverted (hepatitis B surface antigen and hepatitis B antigen negative) may be included in this study after consultation with the Medical Monitor; (c) Primary sclerosing cholangitis; (c) Alcoholic liver disease; (d) Definite autoimmune liver disease or overlap hepatitis; (e) NASH; (f) Gilbert's Syndrome (due to interpretability of bilirubin levels);
(2) presence of clinical complications of PBC or clinically significant (CS) hepatic decompensation at Screening Visit 1 and 2, including: (a) History of liver transplantation; (b) Current placement on a liver transplant list, although subjects who are placed on a transplant list despite a relatively early disease stage (e.g., per regional guidelines) may be eligible as long as they do not meet any of the other exclusion criteria; (c) Current CP Grade B or C (i.e., CP score >6); (d) Portal hypertension with complications, including known gastric or large esophageal varices, poorly controlled or diuretic-resistant ascites, history of variceal bleeds or related therapeutic or prophylactic interventions (e.g., beta blockers, insertion of variceal bands or transjugular intrahepatic portosystemic shunts [TIPS]), or hepatic encephalopathy; (e) Cholangitis with complications, including history or presence of spontaneous bacterial peritonitis, hepatocellular carcinoma, or bilirubin>2×ULN;
(3) medical conditions that may cause nonhepatic increases in ALP (e.g., Paget's disease) or which may diminish life expectancy to <2 years, including known cancers (except carcinomas in situ or other stable, relatively benign conditions);
(4) presence of any other disease or condition that interferes with the absorption, distribution, metabolism, or excretion of drugs including bile salt metabolism in the intestine (e.g., inflammatory bowel disease or gastric bypass procedure [gastric lap band is acceptable]);
(5) current or history of gallbladder disease with or without cholelithiasis and symptoms;
(6) history of drug-induced myopathy;
(7) severe renal failure (serum creatinine>1.5 mg/100 mL (>135 μmol/L); creatinine clearance<60 mL/min) or undergoing dialysis;
(8) platelet count<100 000/ml at Screening Visits 1 and 2;
(9) known history of human immunodeficiency virus (HIV) infection;
(10) history or presence of clinically concerning cardiac arrhythmias likely to affect survival during the study, or Screening (pretreatment) QT or QTc interval of >500 milliseconds;
(11) severe pruritus or required systemic treatment for pruritus (e.g., with bile acid sequestrants [BAS] or rifampicin) within 2 months of Day 1;
(12) history of known or suspected CS hypersensitivity to OCA, BZF, or other fibrates or any of their components;
(13) known photoallergic or phototoxic reactions to fibrates;
(14) if female, known pregnancy, or has a positive urine pregnancy test (confirmed by a positive serum pregnancy test), or lactating;
(15) other CS medical conditions that are not well controlled or for which medication needs are anticipated to change during the study (e.g., type 2 diabetes mellitus, hypothyroidism, nephritic syndrome, dysproteinemia, obstructive liver disease);
(16) treatment with the following medications 30 days before Day 1 or plans to use these medications during the study: azathioprine, colchicine, cyclosporine, methotrexate, mycophenolate mofetil, pentoxifylline, statins, budesonide and other systemic corticosteroids, monoamine oxidase inhibitors (MAOIs), and potentially hepatotoxic drugs (including α-methyl-dopa, sodium valproic acid, isoniazide, and nitrofurantoin);
(17) treatment with the following medications 12 months before Day 1 or plans to use these medications during the study: antibodies or immunotherapy directed against interleukins or other cytokines or chemokines;
(18) participation in another investigational product, biologic, or medical device study within 30 days before Screening;
(19) treatment with commercially available OCA or participation in a previous study involving OCA within 6 months before Screening;
(20) unable to tolerate BZF or other fibrates, treatment with commercially available fibrates or participation in a previous study involving fibrates within 3 months before Screening;
(21) history of or ongoing alcohol or drug abuse within 1 year before Day 1;
(22) history of noncompliance with medical regimens, or is considered by the investigator not able to meet the requirements as specified in the protocol at the Screening visits and throughout the duration of the study;
(23) blood or plasma donation within 30 days before Day 1;
(24) mental instability or incompetence, such that the validity of informed consent or ability to be compliant with the study is uncertain;
(25) a CK value at Screening>5×ULN or any abnormal laboratory value that is considered CS in the opinion of the investigator; and
(26) known or suspected nephrotic syndrome based on the following diagnostic criteria: (a) Proteinuria, spot urine protein: albumin/creatinine ratio of >300-350 mg/mmol; (b) Serum albumin<25 g/l; (c) Clinical evidence of peripheral edema; (d) Severe hyperlipidemia (total cholesterol above>10 mmol/l).

The following exclusion criteria will only apply for subjects who are participating in the HepQuant SHUNT procedure: (1) subjects with a history of known or suspected hypersensitivity to any ingredient in human albumin preparations; (2) subjects with uncontrolled hypertension (defined as a diastolic blood pressure of 110 mmHg or higher); (3) subjects with extensive resection of large segments of small intestine (short gut) or severe gastroparesis; and (4) subjects on either a non-selective beta blocker or an angiotensin-converting enzyme (ACE) inhibitor or angiotensin II receptor blocker (ARB) who are unwilling to delay taking their normal dose the morning of their testing.

### Outcomes/End Points

Primary End Point (can be repeated as necessary): reduction in ALP from baseline in the double-blind treatment period. This end point is evaluated at week 12. Secondary End Point (can be repeated as necessary): The secondary objectives are to assess the effects of the combination of OCA and BZF in comparison to OCA alone in subjects with PBC who have an inadequate response or who are unable to tolerate UDCA on the following: (a) safety and tolerability; (b) response and normalization rates of biochemical disease markers; (c) disease-specific symptoms as assessed by health-related quality of life questionnaires; and (d) biomarkers of bile acid synthesis and homeostasis. This end point is evaluated at the end of the study.

### Screening Period

Subjects will be screened for a period of 2 to 8 weeks before being randomized into the study to allow for the collection of repeat serum chemistry samples (at least 2 weeks apart) for verification of inclusion/exclusion criteria and to establish baseline.

### DB Treatment Period (at least 12 Weeks)

Subjects who meet the entry requirements will be randomized in a 1:1:1:1 ratio on Day 1 to receive either Treatment A (BZF 200 mg IR once daily [QD]), Treatment B (BZF 400 mg SR tablet QD), Treatment C (OCA 5 mg→10 mg QD+BZF 200 mg IR QD), or Treatment D (OCA 5 mg→10 mg QD+BZF 400 mg SR QD). Subjects who are randomized to combination groups will receive OCA 5 mg QD from Day 1 to the day before the Week 4 Visit, followed by OCA 10 mg QD from the Week 4 Visit through the end of the study. To preserve the study blind, appearance-matched placebo tablets for OCA and/or BZF will be administered to subjects in each treatment group from Day 1 to Week 12 as shown in the study design diagrams for the double-blind and LTSE treatment periods. Subjects will be maintained on double-blind dose and transition to the LTSE on the same dose. During the LTSE period, a new dose may be implemented after the review of safety and efficacy data.

Randomization will be stratified at baseline by the total bilirubin levels (≤0.7× or >0.7× upper limit of normal [ULN]) but <2×ULN and ALP (>1.5×ULN but ≤1.67×ULN or >1.67×ULN). The number of subjects with baseline ALP>1.5×ULN but ≤1.67×ULN will not exceed 25% of subjects enrolled in the study.

After the Day 1 Visit, subsequent clinic visits during the double-blind treatment period will occur at approximately Weeks 4, 8, and 12 and then every 12 weeks for the assessment of efficacy, safety, tolerability, and PK. Subjects will also be contacted by telephone at Weeks 2 and 6 (±5 days) to assess for occurrence of any adverse events (AEs), changes to concomitant medications and/or new medications that have been initiated, and medical/surgical procedures, and to verify that the subject is dosing as directed. An evaluation of available efficacy and safety data may occur periodically during both the double-blind and long-term safety extension (LTSE) phases.

### Long-Term Safety Extension (LTSE) Period (up to 48 Weeks)

Subjects will transition to the LTSE phase upon completion of the double-blind phase and will continue the original treatment assignment allocated during the double-blind period. During the LTSE period, the dose of both OCA and BZF may be optimized based on an assessment of safety and efficacy during the double-blind phase, in which case the protocol will be amended, and subjects will be transitioned to the dose selected for further development. All site staff will maintain study blind. Safety and laboratory assessments will be evaluated at clinic visits once every 12 weeks and up to Week 48.

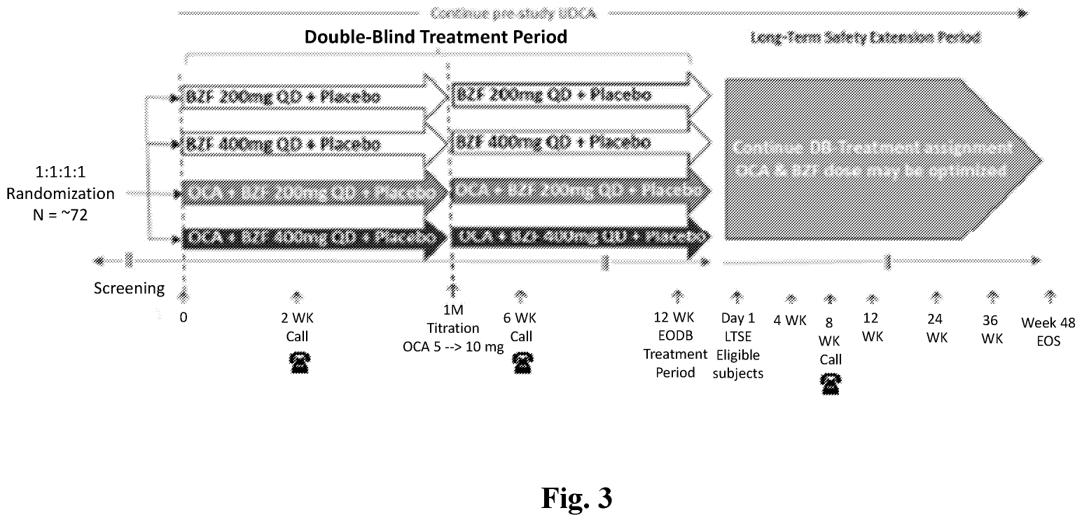

The study design for the double-blind and LTSE treatment periods is shown in FIG. 3, where BZF=bezafibrate; DB=double-blind; EODB=end of DB; EOS=end of study/end of LTSE Period; LTSE=long-term safety extension; OCA=obeticholic acid; QD=once daily; UDCA=ursodeoxycholic acid. Placebo=either OCA or BZF tablets. Note: 1. The Screening Period is 2 weeks to a maximum of 8 weeks; 2. Subjects taking UDCA at the time of enrollment will remain on their stable dose of UDCA during the study; 3. DB dose will continue into the LTSE phase; 4. LTSE Day 1 will be the last visit in the DB Treatment Period (Week 12).

### Study Duration

The total duration of treatment per subject will be a minimum of approximately 68 weeks, which includes up to 20 weeks (8-week Screening Period +12-week double-blind period), followed by 48 weeks of treatment during the LTSE period.

### Number of Subjects

Up to 72 subjects (18 per group) will be enrolled and randomized in a 1:1:1:1 ratio to 1 of 4 treatment arms (Treatments A:B:C:D).

### Dosing Regimen

Subjects will be randomly assigned in a 1:1:1:1 ratio to receive one of the following treatments during the double-blind treatment period:

| Treatment Arm | DB Treatment Morning Dose Regimen |
|---|---|
| Treatment A: | OCA 5 mg placebo tablet (Weeks 0 to 4) |
| BZF 200 mg IR tablet QD | OCA 10 mg placebo tablet (Week 4 to end of DB |
| | treatment) |
| | BZF 200 mg IR tablet |
| | BZF 400 mg SR placebo tablet |
| Treatment B: | OCA 5 mg placebo tablet (Weeks 0 to 4) |
| BZF 400 mg SR tablet QD | OCA 10 mg placebo tablet (Week 4 to end of DB |
| | treatment) |
| | BZF 400 mg SR tablet |
| | BZF 200 mg IR placebo tablet |
| Treatment C: | OCA 5 mg tablet (Weeks 0 to 4) |
| OCA 5 mg → 10 mg QD + | OCA 10 mg tablet (Week 4 to end of DB treatment) |
| BZF 200 mg IR QD | BZF 200 mg IR tablet |
| | BZF 400 mg SR placebo tablet |
| Treatment D: | OCA 5 mg tablet (Weeks 0 to 4) |
| OCA 5 mg → 10 mg QD + | OCA 10 mg tablet (Week 4 to end of DB treatment) |
| BZF 400 mg SR QD | BZF 200 mg IR placebo tablet |
| | BZF 400 mg SR tablet |

BZF = bezafibrate; DB = double-blind; IR = immediate release; OCA = obeticholic acid; QD = once daily; SR = sustained release

All randomized subjects will enter the 12-week double-blind treatment period and will transition to the LTSE phase upon completion of the double-blind phase and will continue the original treatment assignment allocated during the double-blind period. If subjects transition to LTSE prior to the interim analysis, they will continue the original treatment assignment allocated during the double-blind period and maintain blinding to treatment assignment. During the LTSE period, the dose may be optimized based on an assessment of safety and efficacy during the double-blind phase, in which case the protocol will be amended, and subjects will be transitioned to the optimized dose.

### Monitoring and Management of Potential Hepatic Injury and/or Disease Progression

Given the chronic and progressive nature of PBC, it is important to monitor for potential hepatic injury, disease progression, and/or hepatic decompensation. Child-Pugh and MELD scores are reviewed at each visit where labs are drawn. Child Pugh Scores are only applied in patients who have evidence of cirrhosis at screening or demonstrate evidence of cirrhosis at screening or progression to cirrhosis during the study based on known criteria. In addition, adverse events (AEs), signs and symptoms of potential hepatic injury or decompensation, and laboratory values are to be reviewed at regular intervals. Based on the assessments of signs and symptoms of hepatic injury and liver biochemistry, the investigational product (OCA or BZF) may be interrupted or discontinued.

### Dosage Adjustment Criteria

Double-Blind Period—With the exception of the planned dose of 5 mg OCA from Week 1 through Week 4 in the combination treatment groups, dosages for OCA should be maintained constant during the study. However, dose frequency may be modified for the management of pruritus or other safety findings. In the event of tolerability issues such as pruritus or myalgia, the dosing frequency may be decreased at the discretion of the investigator. Subjects can be discontinued from the investigational product by the investigator at any time for clinical safety concerns.

LTSE Period—All eligible, randomized subjects will enter the 12-week double-blind treatment period and transition to the LTSE phase upon completion of the double-blind phase and continue the original treatment assignment allocated during the double-blind phase. During the LTSE period, the dose may be optimized based on an assessment of safety and efficacy during the double-blind phase (interim analysis), in which case the protocol will be amended, and subjects will be transitioned to the optimized dose. Dose frequency may be modified for the management of pruritus or other safety findings. In the event of tolerability issues such as pruritus, the dosing frequency may be decreased at the discretion of the investigator.

### Overview of Assessments

Criteria for Evaluation:

| | |
|---|---|
| Analysis Variables | Endpoint Assessments |

Primary Objectives

| | |
|---|---|
| To assess the effects of the combination of | Reduction in ALP from baseline to Week 12 in |
| OCA and BZF in comparison to BZF | the DB Treatment Period |
| alone in subjects with PBC who have an | |
| inadequate response or who are unable to | |
| tolerate UDCA on ALP. | |

Secondary Objectives

| | |
|---|---|
| Safety and tolerability | SAEs, TEAEs, clinical laboratory assessments, |
| | physical examinations, ECGs, and vital signs, |
| | early discontinuations, and eGFR |
| Response and normalization rates of | ALP response rates of 10%, 20%, and 40% |
| biochemical disease markers | reduction from baseline to Week 12 and |
| | normalization at Week 12 |
| | Clinical laboratory values: |
| | ALP, GGT, ALT, AST, and total and |
| | conjugated bilirubin, APRI |
| Disease-specific symptoms assessed by | PBC-40, pruritus VAS, EQ-5D-5L, and SF-36 |
| health-related quality of life questionnaires | |
| Biomarkers of bile acid synthesis and | C4 and bile acids |
| homeostasis | |

Additional Objectives

| | |
|---|---|
| Noninvasive assessments of liver fibrosis | TE, ELF, and markers of collagen formation and |
| | degradation (Pro-C3, Proc-C5, C3M, and C4M) |
| Noninvasive assessments of liver function | HepQuant |
| Estimated long-term prognosis | GLOBE and UK-PBC scores |
| Safety | MELD (model of end-stage liver disease) score, |
| | physical examinations, ECGs, and vital signs |
| PK parameters | Cₘₐₓ, Tₘₐₓ, AUC, t_{1/2} of BZF, BZF-glucuronide, |
| | BZF-hydroxide, OCA, glycol-OCA and tauro-OCA |
| PK/PD and PK/safety relationships | PK parameters compared to PD parameters and |
| | safety and tolerability assessments (above) |

ALP = alkaline phosphatase; ALT = alanine aminotransferase; APRI = aspartate aminotransferase to platelet ratio index; AST = aspartate aminotransferase; AUC = area under the concentration-time curve; BZF = bezafibrate; C4 = 7α-hydroxy-4-cholesten-3-one; Cmax = peak (maximum) plasma concentration; ECG = electrocardiogram; eGFR = estimated glomerular filtration rate; GGT = gamma-glutamyl transferase; MELD = model of end-stage liver disease; OCA = obeticholic acid; PBC = primary biliary cholangitis; PD = pharmacodynamics; PK = pharmacokinetic; SAE = serious adverse event; t½ = half-life; TE = transient elastography; TEAE = treatment-emergent adverse event; Tmax = time to Cmax; UK = United Kingdom; VAS = visual analog scale.

### Analysis Populations

Evaluable Population—all subjects who complete the DB Treatment Period and have adequate exposure to investigational products (OCA and/or BZF) without any major protocol deviations.

ITT (Intent-to-Treat) Population—all randomized subjects. Treatment assignment is based on the randomized treatment.

mITT Population—all randomized subjects who have baseline and at least one post baseline ALP assessment. Treatment assignment is based on the randomized treatment.

Per-Protocol Population—all subjects in ITT Population without any major protocol deviations. Treatment assignment is based on the randomized treatment.

Safety Population—all randomized subjects who receive at least 1 dose of OCA and/or BZF. Treatment assignment is based on the treatment actually received.

Pharmacokinetic Population—all subjects who receive OCA and/or BZF and have at least 1 confirmed analyzable sample. Subjects must not have any major protocol deviations that potentially affect exposure levels.

LTSE (long-term safety extension) Population—all subjects who receive at least 1 dose of OCA and/or BZF during the LTSE period.

### Efficacy Analyses

Primary Efficacy Analysis: The mITT Population will be the primary population used for the primary efficacy analyses. The primary efficacy endpoint is the change in ALP from baseline to Week 12 in the double-blind treatment period. Analyses of change in ALP will be carried out using an analysis of covariance (ANCOVA) model at Week 12 with change from baseline as the dependent variable, treatment group and randomization stratification factor as fixed effects, and the baseline values as a covariate. The same analysis will be carried out using percent change from baseline as the dependent variable. The primary efficacy analyses will also be conducted in the Per-Protocol Population.

Secondary and Additional Efficacy Analyses: The ITT Population will be the primary population used for the secondary and additional efficacy analyses. The secondary and additional efficacy analyses will not be analyzed in the Per-Protocol Population. The secondary and additional efficacy endpoints include: (a) The response rates of 10%, 20%, and 40% change and normalization rates at Week 12; (b) Change from baseline of PBC-40, pruritus VAS, EQ-5D-5L, and SF-36 at Week 12; (c) Change from baseline of ALP, GGT, ALT, AST, and total and conjugated bilirubin; AST to platelet ratio index [APRI] at Week 12; (d) Change from baseline of TE, ELF and markers of collagen formation and degradation (Pro-C3, Pro-C5, C3M, and C4M) at Week 12; (e) Change from baseline of GLOBE and UK-PBC scores at Week 12; and (f) Change from baseline of liver disease severity index (DSI) from HepQuant-SHUNT test at Week 12. Secondary and additional efficacy analyses will be summarized using descriptive statistics at Baseline and at each scheduled post-baseline visit comparing the BZF and OCA +BZF treatment groups. Analyses for ALP response rates of 10%, 20%, and 40% change and normalization rates will be performed using a Cochran-Mantel-Haenszel test stratified by the randomization stratification factor. Analyses of PBC-40, pruritus VAS, EQ-5D-5L, and SF-36 will be performed using a Wilcoxon rank-sum test. Liver function evaluated by HepQuant-SHUNT will be summarized with descriptive statistics at baseline and post-baseline visits. Further analysis details will be specified in the statistical analysis plan (SAP) and/or a separate clinical pharmacology analysis plan.

### Pharmacokinetic Analyses

The PK Population will be the primary population used for PK, PK/PD, and PK/safety analyses. PK parameter estimates will be determined for plasma BZF and unconjugated OCA (parent), glyco-OCA, tauro-OCA, and total OCA (sum of OCA, glyco-OCA, and tauro-OCA) using standard noncompartmental methods based on actual sample collection times.

### PK/PD and PK/Safety Analyses

The PK/PD relationship of C4, total endogenous bile acids, and ALP as a function of total OCA and/or BZF PK exposure parameters will be evaluated. The PK/PD relationship of pruritus and other safety indicators, such as liver biochemistry markers (e.g., ALP), as a function of total OCA and/or BZF PK exposure parameters will be evaluated.

### Safety Analyses

The Safety Population is the primary population used for safety analyses. Treatment assignment is based on the treatment actually received. Safety data, including serious AEs (SAEs), treatment-emergent AEs (TEAEs), physical examinations, electrocardiograms (ECGs), vital signs, clinical laboratory assessments, and treatment discontinuations are compared across all treatment groups during the DB Treatment Period.

The incidence of TEAEs and SAEs is tabulated by System Organ Class (SOC) and Preferred Term for each treatment group and similarly by severity and relationship to treatment.

Laboratory parameters and vital signs are summarized by treatment group using descriptive statistics at Baseline and at each scheduled postbaseline visit. The change from baseline is also summarized. ECGs are summarized by treatment group using frequency at each visit. The shift from baseline is also summarized. Baseline is defined as the mean of all available evaluations before treatment.

### LTSE Analyses

Similar analyses to that described for the double-blind treatment period will be conducted for the LTSE Period data using the double-blind baseline value with the exception of PK, which will not be performed during the LTSE Period. Analyses based on the double-blind baseline will be performed using randomized treatment groups.

### Interim Analysis

An interim analysis will be performed to guide decision-making for the phase 3 trial. No futility or superiority stopping rules will apply for the interim analysis. The interim analysis will be carried out when approximately 10 subjects per treatment group complete the double-blind treatment period of the study. In addition to the routine safety review, the DMC will also review the interim analysis. The change in ALP from baseline to Week 12 in the double-blind treatment period using the mITT population will be analyzed in the interim analysis. When the Cohen's D effect sizes of change from baseline of ALP are ≥0.93 at Week 12 between OCA 5-10 mg+BZF 200mg QD arm (Treatment B) and BZF 200 mg QD arm, or between OCA 5-10 mg+BZF 400 mg QD arm (Treatment D) and BZF 400 mg QD arm, the Treatment B or D will be determined to be effective during the interim analysis.

### Sample Size Justification

A sample size of 18 subjects per treatment group will provide at least 80% power to detect a treatment difference for change in ALP of −60 U/L, assuming that the mean absolute changes in ALP for OCA+BZF and OCA treatment groups are approximately 160 and 100 U/L, respectively, with a pooled standard deviation of 58 U/L and a 10% dropout rate, based on a 2 sided independent 2 group t test at an alpha level of 0.05.

### Example 3: HepQuant-SHUNT to Measure Liver Function to Assess to Assess Liver Disease and Treatment Effect

Hepatic inflammation and hepatic fibrosis impair hepatocyte function and hepatic perfusion. Evolution to cirrhosis is associated with increasing hepatic impairment—ultimately leading to portal hypertension and portal-systemic shunting. Portal hypertension (PH) is a risk factor for poor outcome in liver disease.

The HepQuant SHUNT test is an assay that is included as an additional study objective. This Example describes the HepQuant SHUNT test and its use for assessing liver disease and treatment effects in this study. The HepQuant tests measure the clearance of cholates labeled with molecular probes (carbon-13 [¹³C], and deuterium [4D]). In brief, the test involves placement of an indwelling peripheral venous catheter (usually in the antecubital vein of the arm), an injection of ⁿC-cholate (cold, stable label, NOT RADIOACTIVE) intravenously, and a drink of flavored solution of 40 mg d4-cholate (d4-CA or 4D-CA) (again, cold, stable label, NOT RADIOACTIVE). Blood samples will be taken at predose of cholate and 5, 20, 45, 60, 90 minutes post-dosing. Blood samples will be allowed to clot and be spun, and the serum will be transferred to transport tubes for mailing to HepQuant lab for processing and analysis. HepQuant SHUNT tests are capable of monitoring hepatocellular function, total hepatic perfusion, portal inflow to the liver, and portal-systemic shunting. Similar to HVPG, HepQuant SHUNT assesses the portal circulation, but is non-invasive with high subject tolerability and lower cost.

Liver diseases alter hepatocyte function and the portal circulation which manifests as portal hypertension and portal-systemic shunting. The clinical consequences are coagulopathy, jaundice, varices, ascites and encephalopathy. As liver disease progresses, from early stage with minimal fibrosis to late stage fibrosis, cirrhosis, and clinical complications, hepatic function and the two circulatory inflows to the liver, systemic and portal, become increasingly impaired. The HepQuant-SHUNT test measures a liver-specific function, clearance of cholate, from both systemic and portal circulations simultaneously. The test is based on the fact that liver disease impairs function and alters the portal circulation. As blood flow to the liver becomes impaired, a greater amount of the administered cholates escapes extraction by the liver and spills over into the systemic circulation; this is manifested as an increase in systemic cholate concentration in the blood samples obtained through the peripheral venous catheter. HepQuant SHUNT quantifies the changes in liver function and the portal circulation from early through late stages of disease.

In prior studies of chronic hepatitis C, the Disease Severity Index (DSI) from the HepQuant SHUNT test correlated with ISHAK and METAVIR stages of fibrosis and predicted likelihood of cirrhosis, varices, and risk for clinical outcome. DSI has performed similarly in patients with chronic hepatitis C, NAFLD, and PSC. The HepQuant study is to compare the change in DSI between treatment arms at each time point over the time period of the study.

### Test Administration

The HepQuant SHUNT test is performed after at least 5 hours of fasting, usually after an overnight fast, and requires venous access via a standard indwelling peripheral venous catheter, preferably placed in the antecubital fossae. Approximately 3 mL blood samples are obtained at baseline and at 5, 20, 45, 60, and 90 minutes after dosing with the cholate solutions; and, ≥1 mL serum is shipped at ambient temperature to the HepQuant laboratory for analysis of cholate concentrations. The subject may be in a bed seated upright or in a recliner chair—the subject should be seated in an upright position, or if in bed, have the head of the bed elevated by at least 30 degrees to aid gastric emptying of the orally administered dose of 4D-CA solution.

Prior to administration, the HepQuant SHUNT Liver Diagnostic Kits are kept at ambient temperature. For the oral 4D-CA dose, the full contents of the d4-CA solution are poured into a 40 mL cup and flavoring added. For the intravenous ¹³C-CA dose, 5 mL (from a total of 5.5 mL) are removed from the ¹³C-CA solution vial and mixed with 5 ml of the albumin solution (25% w/v human serum albumin, USP grade, GRIFOLS). The ¹³C-CA/Albumin mixture is injected intravenously over 1 minute by the person administering the test. The 4D-cholate/flavoring mixture is administered orally simultaneously over the same minute.

The test can be administered in one of two methods: (1) the Two-Arm method and (2) the Single-Arm, Single-Catheter method. The Two-Arm method uses the intravenous (IV) catheter solely for blood sampling. A separate butterfly or small catheter, placed in the opposite arm, is used for injection of the IV cholate/albumin solution. The Two-Arm method is the preferred method of administration. The Single-Arm, Single-Catheter method uses the same catheter for both injection of the IV cholate/albumin and subsequent blood sampling. A strict flushing procedure should be used if the Single-Arm, Single-Catheter is used—to avoid carryover of the injected cholate solution into the subsequent blood samples. If the subject were to experience an anaphylactic or hypersensitivity reaction to the compounds, administration should be stopped, and the subject should be treated in accordance with standard of care. The subject should not undergo any future HepQuant tests, but may remain enrolled in the parallel drug study at the discretion of the investigator.

### Test Outputs

Cholate concentrations (endogenous unlabeled CA, ¹³C-CA, and d4-CA) will be measured from the timed serum samples (0, 5, 20, 45, 60, and 90 minutes) and concentrations of each labeled cholate as a function of time will be modeled as a spline curve in order to calculate the area under curve (AUC). The HepQuant SHUNT test parameters are:
Systemic HFR: The intravenous clearance (Clᵢᵥ, mL min⁻¹) is defined as the dose/AUC for ¹³C-CA. The Systemic HFR is defined as the Clᵢᵥ per kg of body weight and is expressed as mL min⁻¹kg⁻¹.
Portal HFR: The apparent oral clearance (Clₒᵣₐₗ, mL min⁻¹) is defined as the dose/AUC for d4-cholate. The Portal HFR is defined as the Clₒᵣₐₗ per kg of body weight and is also expressed as mL min-⁻¹ kg-₋₁.
SHUNT: SHUNT, the portal-systemic shunt fraction, is calculated as the ratio Systemic HFR/Portal HFR×100%.
DSI: The calculation for Disease Severity Index is according to a formula derived from Systemic HFR and Portal HFR.
STAT: The serum concentration of d4-cholate from the 60-minute blood sample correlates well with DSI (r2=0.88) and is independently analyzed for links to disease severity and treatment effects, similar to the analyses for DSI.

### Known Potential Risks

The risks associated with the test compounds include: (1) Allergic reaction to cholate compounds (theoretical—none yet reported); and (2) Allergic reaction to human serum albumin (HSA), where reactions could include: (a) rash; (b) having a hard time breathing;(c) wheezing when you breathe; (d) sudden drop in blood pressure; (e) swelling around the mouth, throat, or eyes; (f) fast pulse; (g) sweating; (h) severe reactions are very rare but a severe reaction (called anaphylaxis); and (i) can lead to profoundly low blood pressure and even death.

The risks associated with the indwelling catheter include: (1) Pain with placement of catheter; (2) Thrombosed vein; and (3) Hematoma.

The risks associated with phlebotomy include: (1) Localized pain; (2) Bruising; (3) Occasional lightheadedness; (4) Fainting; and (5) Infection at the site (rare).

The risks associated with fasting include: (1) Dizziness; (2) Headache; (3) Stomach Discomfort; and (4) Fainting.

### Test Compounds

Cholates, labeled with stable (nonradioactive) isotopes, occur naturally and are not known to have any deleterious or adverse effects when given intravenously or orally in the doses used in HepQuant (HQ) tests. The serum cholate concentrations that are achieved by either the intravenous or oral doses are similar to the serum concentrations of bile acids that occur after the ingestion of a fatty meal.

The two cholates used in the HepQuant test for this study are labeled with stable (non-radioactive) forms of carbon and hydrogen that are found in nature and can be measured in blood. These forms of cholate have been used with FDA INDs (65121 and 65123) since 2002, and their use in humans has been monitored since that time.

### Analysis of Results

Liver function evaluated by HepQuant-SHUNT will be summarized with descriptive statistics at baseline and post-baseline visits. The major objective of this HepQuant SHUNT study is to determine whether serial changes in DSI indicate a treatment effect, and to define the relationship of change in DSI to change in other measures of treatment response. Further analysis details will be specified in the SAP and/or a separate clinical pharmacology analysis plan. For responder analyses using DSI as the endpoint, a significant treatment response in a given subject will be defined as a two point or greater decrease in DSI.

1. A method for preventing, ameliorating or treating a cholestatic liver disease, comprising administering to a patient in need thereof a pharmaceutical composition comprising a combination of an FXR agonist and a fibrate, and optionally one or more pharmaceutically acceptable carriers.

2. The method of claim 1, wherein the FXR agonist is of formula A: 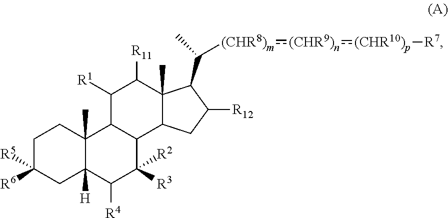
or a pharmaceutically acceptable salt, solvate, amino acid, sulfate or glucuronide conjugate, or prodrug thereof, wherein:
R¹ is OH, alkoxy, or oxo;
R² and R³ are each independently H, OH, OSO₃H, OCOCH₃, OPO₃H₂, halogen, or alkyl optionally substituted with one or more halogen or OH, or R² and R³ taken together with the carbon atom to which they are attached form a carbonyl;
R⁴ is H, halogen, alkyl optionally substituted with one or more halogen or OH, alkenyl, or alkynyl;
R⁵ and R⁶ are each independently H, OH, OSO₃H, OCOCH₃, OPO₃H₂, halogen, or alkyl optionally substituted with one or more halogen or OH, or R⁵ and R⁶ taken together with the carbon atom to which they are attached form a carbonyl;
R⁷ is OH, OSO₃H, SO₃H, OSO₂NH₂, SO₂NH₂, OPO₃H₂, PO₃H₂, CO₂H, C(O)NHOH, NH(CH₂)₂SO₃H, NHCH₂CO₂H, tetrazolyl, oxadiazolyl, thiadiazolyl, 5-oxo-1,2,4-oxadiazolyl, 5-oxo-1,2,4-thiadiazolyl, oxazolidine-dionyl, thiazolidine-dionyl, 3-hydroxyisoxazolyl, 3 -hydroxyisothiazolyl, pyrimidine, 3,5-difluoro-4-hydroxyphenyl or 2,4-difluoro-3 -hydroxyphenyl;
R⁸, R⁹, and R¹⁰ are each independently H, OH, halogen, or alkyl optionally substituted with one or more halogen or OH, or R⁸ and R⁹ taken together with the carbon atoms to which they are attached form a 3- to 6-membered carbocyclic or heterocyclic ring comprising 1 or 2 heteroatoms selected from N, O, and S, or R⁹ and R¹⁰ taken together with the carbon atoms to which they are attached form a 3- to 6-membered carbocyclic or heterocyclic ring comprising 1 or 2 heteroatoms selected from N, O, and S;
R₁₁ and R₁₂ are each independently H or OH;
m is 0, 1, or 2;
n is 0 or 1; and
p is 0 or 1.

3. The method of claim 1 or 2, wherein the FXR agonist of formula A is of formula 1: 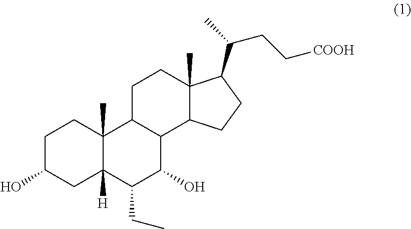 [SMILES: CC[C@H]1[C@@H](O)C2C3CC[C@H]([C@H](C)CCC(=O)O)[C@@]3(C)CCC2[C@@]2(C)CC[C@@H](O)C[C@@H]12]
or a pharmaceutically acceptable salt or amino acid conjugate thereof.

4. The method of claim 1 or 2, wherein the FXR agonist of formula A is of formula 2: 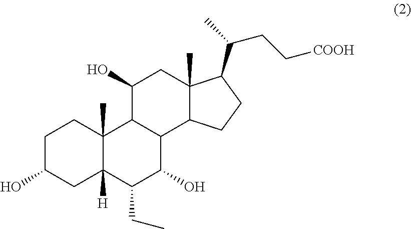 [SMILES: CC[C@H]1[C@@H](O)C2C3CC[C@H]([C@H](C)CCC(=O)O)[C@@]3(C)C[C@H](O)C2[C@@]2(C)CC[C@@H](O)C[C@@H]12]
or a pharmaceutically acceptable salt or amino acid conjugate thereof.

5. The method of claim 1 or 2, wherein the FXR agonist of formula A is of formula 3: 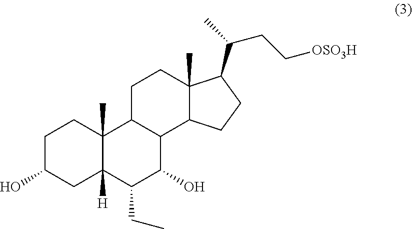
or a pharmaceutically acceptable salt thereof.

6. The method of any one of claims 1, 2, and 5, wherein the FXR agonist of formula A is Compound 3a or Compound 3b:

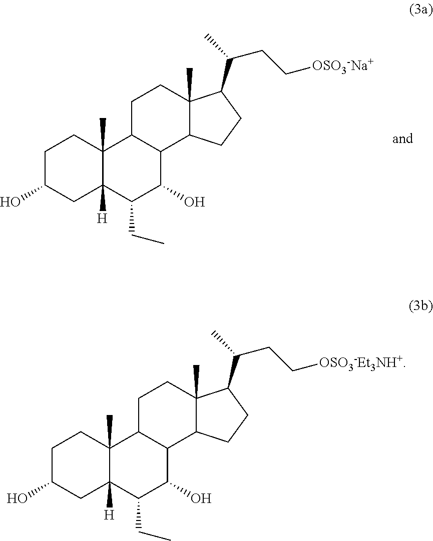

7. The method of any one of claims 1-3, wherein the FXR agonist of formula A is obeticholic acid (OCA) or a pharmaceutically acceptable salt or amino acid conjugate thereof.

8. The method of any one of claims 1-7, wherein the fibrate is bezafibrate.

9. The method of any one of claims 1-8, wherein the cholestatic liver disease is primary biliary cholangitis.

10. A method for treating a cholestatic liver disease in a patient in need thereof, comprising administering to the patient a composition comprising obeticholic acid (OCA) or a pharmaceutically acceptable salt or amino acid conjugate thereof in the amount of 5-50 mg and bezafibrate in the amount of 200-400 mg, wherein the composition is administered once daily (QD).

11. A method for treating PBC in a patient in need thereof, comprising administering to the patient obeticholic acid (OCA) or a pharmaceutically acceptable salt or amino acid conjugate thereof in the amount of 5-50 mg once daily (QD) and bezafibrate in the amount of 200-400 mg QD.

12. The method of any one of claims 7-11, wherein OCA or a pharmaceutically acceptable salt or amino acid conjugate thereof is administered in the amount of 5-50 mg.

13. The method of any one of claims 7-12, wherein OCA or a pharmaceutically acceptable salt or amino acid conjugate thereof is administered in the amount of 5 mg.

14. The method of any one of claims 7-12, wherein OCA or a pharmaceutically acceptable salt or amino acid conjugate thereof is administered in the amount of 10 mg.

15. The method of any one of claims 7-14, wherein bezafibrate is administered in the amount of 200 mg.

16. The method of any one of claims 7-14, wherein bezafibrate is administered in the amount of 400 mg.

17. The method of any one of claims 1-16, further comprising a step of assessing, monitoring, measuring, or detecting liver function.

18. The method of claim 17, wherein the step of assessing, monitoring, measuring, or detecting liver function comprises performing a non-invasive assay.

19. The method of claim 18, wherein the non-invasive assay is a HepQuant SHUNT assay.

20. The method of any one of claims 1-19, wherein the patient has an inadequate response to, or is intolerant to, ursodeoxycholic acid treatment.

21. A composition comprising obeticholic acid (OCA) or a pharmaceutically acceptable salt or amino acid conjugate thereof in the amount of 5-50 mg and bezafibrate in the amount of 200-400 mg for use in the treatment of PBC, wherein the composition is for administration once daily.

22. Use of a composition comprising obeticholic acid (OCA) or a pharmaceutically acceptable salt or amino acid conjugate thereof in the amount of 5-50 mg and bezafibrate in the amount of 200-400 mg in the manufacture of a medicament for the treatment of PBC, wherein the composition is for administration once daily.

23. Obeticholic acid (OCA) or a pharmaceutically acceptable salt or amino acid conjugate thereof for use in combination with bezafibrate in the treatment of PBC, wherein the OCA or a pharmaceutically acceptable salt or amino acid conjugate thereof is for administration in the amount of 5-50 mg once daily (QD) and bezafibrate is for administration in the amount of 200-400 mg QD.

24. Use of obeticholic acid (OCA) or a pharmaceutically acceptable salt or amino acid conjugate thereof in combination with bezafibrate in the manufacture of a medicament for use in the treatment of PBC, wherein the OCA or a pharmaceutically acceptable salt or amino acid conjugate thereof is for administration in the amount of 5-50 mg once daily (QD) and bezafibrate is for administration in the amount of 200-400 mg QD.

25. A combinational therapy for the treatment of PBC, comprising administration of obeticholic acid (OCA) or a pharmaceutically acceptable salt or amino acid conjugate thereof in the amount of 5-50 mg once daily (QD) and bezafibrate in the amount of 200-400 mg QD.
